SEQUENCE LISTING (13 sequences)

SEQ ID NO: 1 - DNA, 4214 bp, Homo sapiens
gcgcctcgggcggcttctcgccgctcccaggtctggctggctggaggagtctcagctctcagccgctcgcccgcccccgctccgggccctcccctagtcgccgctgtggggcagcgcctggcgggcggcccgcgggcgggtcgcctcccctcctgtagcccacacccttcttaaagcggcggcgggaagatgaggcttcgggagccgctcctgagcggcagcgccgcgatgccaggcgcgtccctacagcgggcctgccgcctgctcgtggccgtctgcgctctgcaccttggcgtcaccctcgtttactacctggctggccgcgacctgagccgcctgccccaactggtcggagtctccacaccgctgcagggcggctcgaacagtgccgccgccatcgggcagtcctccggggagctccggaccggaggggcccggccgccgcctcctctaggcgcctcctcccagccgcgcccgggtggcgactccagcccagtcgtggattctggccctggccccgctagcaacttgacctcggtcccagtgccccacaccaccgcactgtcgctgcccgcctgccctgaggagtccccgctgcttgtgggccccatgctgattgagtttaacatgcctgtggacctggagctcgtggcaaagcagaacccaaatgtgaagatgggcggccgctatgcccccagggactgcgtctctcctcacaaggtggccatcatcattccattccgcaaccggcaggagcacctcaagtactggctatattatttgcacccagtcctgcagcgccagcagctggactatggcatctatgttatcaaccaggcgggagacactatattcaatcgtgctaagctcctcaatgttggctttcaagaagccttgaaggactatgactacacctgctttgtgtttagtgacgtggacctcattccaatgaatgaccataatgcgtacaggtgtttttcacagccacggcacatttccgttgcaatggataagtttggattcagcctaccttatgttcagtattttggaggtgtctctgctctaagtaaacaacagtttctaaccatcaatggatttcctaataattattggggctggggaggagaagatgatgacatttttaacagattagtttttagaggcatgtctatatctcgcccaaatgctgtggtcgggaggtgtcgcatgatccgccactcaagagacaagaaaaatgaacccaatcctcagaggtttgaccgaattgcacacacaaaggagacaatgctctctgatggtttgaactcactcacctaccaggtgctggatgtacagagatacccattgtatacccaaatcacagtggacatcgggacaccgagctagcgttttggtacacggataagagacctgaaattagccagggacctctgctgtgtgtctctgccaatctgctgggctggtccctctcatttttaccagtctgagtgacaggtccccttcgctcatcattcagatggctttccagatgaccaggacgagtgggatattttgcccccaacttggctcggcatgtgaattcttagctctgcaaggtgtttatgcctttgcgggtttcttgatgtgttcgcagtgtcaccccagagtcagaactgtacacatcccaaaatttggtggccgtggaacacattcccggtgatagaattgctaaattgtcgtgaaataggttagaatttttctttaaattatggttttcttattcgtgaaaattcggagagtgctgctaaaattggattggtgtgatctttttggtagttgtaatttaacagaaaaacacaaaatttcaaccattcttaatgttacgtcctccccccacccccttctttcagtggtatgcaaccactgcaatcactgtgcatatgtcttttcttagcaaaaggattttaaaacttgagccctggaccttttgtcctatgtgtgtggattccagggcaactctagcatcagagcaaaagccttgggtttctcgcattcagtggcctatctccagattgtctgatttctgaatgtaaagttgttgtgtttttttttaaatagtagtttgtagtattttaaagaaagaacagatcgagttctaattatgatctagcttgattttgtgttgatccaaatttgcatagctgtttaatgttaagtcatgacaatttatttttcttggcatgctatgtaaacttgaatttcctatgtatttttattgtggtgttttaaatatggggaggggtattgagcattttttagggagaaaaataaatatatgctgtagtggccacaaataggcctatgatttagctggcaggccaggttttctcaagagcaaaatcaccctctggccccttggcaggtaaggcctcccggtcagcattatcctgccagacctcggggaggatacctgggagacagaagcctctgcacctactgtgcagaactctccacttccccaaccctccccaggtgggcagggcggagggagcctcagcctccttagactgacccctcaggcccctaggctggggggttgtaaataacagcagtcaggttgtttaccagccctttgcacctccccaggcagagggagcctctgttctggtgggggccacctccctcagaggctctgctagccacactccgtggcccaccctttgttaccagttcttcctccttcctcttttcccctgcctttctcattccttccttcgtctccctttttgttcctttgcctcttgcctgtcccctaaaacttgactgtggcactcagggtcaaacagactatccattccccagcatgaatgtgccttttaattagtgatctagaaagaagttcagccgaacccacaccccaactccctcccaagaacttcggtgcctaaagcctcctgttccacctcaggttttcacaggtgctcccaccccagttgaggctcccacccacagggctgtctgtcacaaacccacctctgttgggagctattgagccacctgggatgagatgacacaaggcactcctaccactgagcgcctttgccaggtccagcctgggctcaggttccaagactcagctgcctaatcccagggttgagccttgtgctcgtggcggaccccaaaccactgccctcctgggtaccagccctcagtgtggaggctgagctggtgcctggccccagtcttatctgtgcctttactgctttgcgcatctcagatgctaacttggttctttttccagaagcctttgtattggttaaaaattattttccattgcagaagcagctggactatgcaaaaagtatttctctgtcagttccccactctataccaaggatattattaaaactagaaatgactgcattgagagggagttgtgggaaataagaagaatgaaagcctctctttctgtccgcagatcctgacttttccaaagtgccttaaaagaaatcagacaaatgccctgagtggtaacttctgtgttattttactcttaaaaccaaactctaccttttcttgttgttttttttttttttttttttttttttttggttaccttctcattcatgtcaagtatgtggttcattcttagaaccaagggaaatactgctccccccatttgctgacgtagtgctctcatgggctcacctgggcccaaggcacagccagggcacagttaggcctggatgtttgcctggtccgtgagatgccgcgggtcctgtttccttactggggatttcagggctgggggttcagggagcatttccttttcctgggagttatgaccgcgaagttgtcatgtgccgtgcccttttctgtttctgtgtatcctattgctggtgactctgtgtgaactggcctttgggaaagatcagagagggcagaggtggcacaggacagtaaaggagatgctgtgctggccttcagcctggacagggtctctgctgactgccaggggcgggggctctgcatagccaggatgacggctttcatgtcccagagacctgttgtgctgtgtattttgatttcctgtgtatgcaaatgtgtgtatttaccattgtgtagggggctgtgtctgatcttggtgttcaaaacagaactgtatttttgcctttaaaattaaataatataacgtgaataaatgaccctatctttgtaacaaaaaaaaaaaaaaa

SEQ ID NO: 2 - protein, 398 aa, Homo sapiens
MRLREPLLSGSAAMPGASLQRACRLLVAVCALHLGVTLVYYLAGRDLSRLPQLVGVSTPLQGGSNSAAAIGQSSGELRTGGARPPPPLGASSQPRPGGDSSPVVDSGPGPASNLTSVPVPHTTALSLPACPEESPLLVGPMLIEFNMPVDLELVAKQNPNVKMGGRYAPRDCVSPHKVAIIIPFRNRQEHLKYWLYYLHPVLQRQQLDYGIYVINQAGDTIFNRAKLLNVGFQEALKDYDYTCFVFSDVDLIPMNDHNAYRCFSQPRHISVAMDKFGFSLPYVQYFGGVSALSKQQFLTINGFPNNYWGWGGEDDDIFNRLVFRGMSISRPNAVVGRCRMIRHSRDKKNEPNPQRFDRIAHTKETMLSDGLNSLTYQVLDVQRYPLYTQITVDIGTPS

SEQ ID NO: 3 - DNA, 4535 bp, Mus musculus
actccattttctcactatcccaaggctaccgcctgtcctcttgtcctcactagctgctgtcccaggcccatacccgagtcactgggtacgtcaggaagtggtgagcagctcgttgaacaagaagctggtttagagagacttaagctccacggtgaacgctaggaagaaaagctgagcgccgctgggtatgggatgcccgctcaacccaaggctgcggtggggctggcggagcgccacgtttagtccccaagtgtcgcgacgccctgcaacccaacctcccgagggcccgccccttagtgcctgccccccaagctccgccctcccttagctgccagaaaaacccggctgaaggttccgcccctcagtcccctctccgagccttgtccctcatgccccgcccctctcgaaaaagacccgctttgcggccccaccccttagactccgcccccccaggttccgcgaacagccttccgaaacccggttgaggccccgccctaggtcgagaccacgccccctgctcatccgcgcttgtggttcccgctgatctctccaaagtcccgcggccaggggtgtcggctttccgccgcaggcggccggtgctgcgctaggtcctagccccctccccccggcccctccttcgatcgctgtggtcgggtagcgcctggcgggcggcctgcgggcgggccgtcctctcagccgtagcccaccccctcttaaagccgcggcgggaagatgaggtttcgtgagcagttcctgggcggcagcgccgcgatgccgggcgcgaccctgcagcgggcctgccgcctgctcgtggccgtctgcgcgctgcacctcggcgtcaccctcgtctattacctctctggccgcgatctgagccgcctgccccagttggtcggagtctcctctacactgcagggcggcacgaacggcgccgcagccagcaagcagcccccaggagagcagcggccgcggggtgcgcggccgccgcctcctttaggcgtctccccgaagcctcgcccgggtctcgactccagccctggtgcagcttctggccccggcttgaagagcaacttgtcttcgttgccagtgcccaccaccactggactgttgtcgctgccagcttgccctgaggagtccccgctgctcgttggccccatgctgattgactttaatattgctgtggatctggagcttttggcaaagaagaacccagagataaagacgggcggccgttactcccccaaggactgtgtctctcctcacaaggtggccatcatcatcccattccgtaaccggcaggagcatctcaaatactggctgtattatttgcatcccatccttcagcgccagcaactcgactatggcatctacgtcatcaatcaggctggagacaccatgttcaatcgagctaagctgctcaatattggctttcaagaggccttgaaggactatgattacaactgctttgtgttcagtgatgtggacctcattccgatggacgaccgtaatgcctacaggtgtttttcgcagccacggcacatttctgttgcaatggacaagttcgggtttagcctgccatatgttcagtattttggaggtgtctctgctctcagtaaacaacagtttcttgccatcaatggattccctaataattattggggttggggaggagaagatgacgacatttttaacagattagttcataaaggcatgtctatatcacgtccaaatgctgtagtagggaggtgtcgaatgatccggcattcaagagacaagaaaaatgagcccaatcctcagaggtttgaccggatcgcacatacaaaggaaacgatgcgcttcgatggtttgaactcacttacctacaaggtgttggatgtacagagatacccgttatatacccaaatcacagtggacatcgggacaccgagatagcatttttagtaccaataagagacctgagaatggccggagacctcagatatgtgtctctgccagttgactgggctggtccctctcatttgtacagtctgaatgacagttcttcttatcattcagacgtccctccagatgcccagggtgagtgtaacatttacccacaacctggctcggcactggatgaaattctacaaggtgagtggagtgtaaaactggtcagcccttggagagacttcttggttgtgtcacccccaaagagtcagaactgtacacagttcaaaacttagtgactgtgggtcacattcccactgttgaaactgctaaattgtgacctggggaaggactttgctttagtcggtgatgttcgtacttggtgacaaattgagactgctgctggattcagattgacaagattttcttggattttttttttatacgaaaatcaaaatttcaatcagtctcgtgctctgtccctttacatcggtatgcgactattacaatcactgtgtgtgtgtcttttcttagcaaaggcgttttaaaacttgagcctggaccttggggtcctgtagtgtgtggattccaaggccttgccctcagagcaggggcctgggcactctcactcacgtggcctgtctccagatccctgtctgatttctgaatgtaaagaggctttttgttttgtttttgtttttgtttttagaagcagttcgtagtatttgaaagaataaatcaagttttgattatgctataggttgatttttgtgttgatccaaatcagaatagctattgagtgtttaagtcatgactttatttttctgggcatgctatataaacttgaatttcctatgtatttttattgtggtattttaaatgtgggggaagggattgggtattttttaggggaaaaaaaataatgtatgctgtagtggtcaaaggagctctgattgagctggcaggccaggttttgtaaagagcaaaattactttcaggtcccttgttgagggaacaggtcagcgttatcctgccagacatctgggtcaggaacttgggagacaacatctgcttacctggcagaactccccactccactcccccaggtgggctgggtggaaggaccctcagccaccaagaactgacgccctcatggccctagactatggggttgaaaactcatagcagtcaggtttgtttaccatctctccgagcctccaccacactccctgacccacccaacgtctccagttcttattgttctccttccttccttctttcctttcccttctcacctctttgcctccttccctaaaattcgctgtggccctcggggtcagactgttcattctccagcatgattgtgccttttaattagaggtctagaaagttcacctgaacccacacccccttcttcctgagaattctggtgcctctaaagcccctccttgccactcagggttttagtgggtgctcccactgctcagtgtacccatgagctgtcgtcattcacagagccctgtgccaggccgtgttctgtgcctgcctgcggtgatgtagccagaagcacgtactcaggtcatcaagccgagagggcgtcaaagaggaagcctgggctgggatccttaggctcagctgtctgtctaatcccagagctaagccctttctgcttcgggcagcctggactccagccctgagtatagaggcggtgtggatggctggccctcatcctacctgtgtctttattgctttgatcaccacagtctctaacttgctgctttctccagaagcctttttatgtatgggttagaaactcttttttccctagtcagagcagccaggctgtacaaaatctgtgctctctcggttccccatgcctctagaaggccgttaaaaccaagactggatgcactgagaggagtctttttttttttttttttttttttctgtcccaagatcctgacttttccaaagtgccttaaaagaagggagacagactcattgagtgtgttgcttctttctctcctaaagcaaacctctgccttttctctatgctgttcgtgttgggttttgttcatgtcagatacgtggttcattctcaggaccaagggaaactgtcttgcctgtcctcctatctcctccctttcagcatcctttggggaacctccagcccaaggcactgttaggcttgggcattagcctggcccaagagatgccactggggactagaggacagaaggggacgttcccttcacctgggaattctcaccctggtgtcaacatgctgtgcctttctctttctctatttttgttggtgacctggatagtgtggcctttgggacagatcagaggcacaggaaagcaaaggaaatgtgccaccctttagcctggcagggtctccctgcccactgggctgttccagacctgtgatactgtgtttgtatgttgatatcccatgtgtacaaatgtgtttaccattggggctgggggtgggtggctgtctctgaccttggcattcagaacagacatctgtatttttgcctttgaaatgcagtaatataacgtgaataaatgaccttatctttgtaactggcttcttttcttttttg

SEQ ID NO: 4 - protein, 399 aa, Mus musculus
MRFREQFLGGSAAMPGATLQRACRLLVAVCALHLGVTLVYYLSGRDLSRLPQLVGVSSTLQGGTNGAAASKQPPGEQRPRGARPPPPLGVSPKPRPGLDSSPGAASGPGLKSNLSSLPVPTTTGLLSLPACPEESPLLVGPMLIDFNIAVDLELLAKKNPEIKTGGRYSPKDCVSPHKVAIIIPFRNRQEHLKYWLYYLHPILQRQQLDYGIYVINQAGDTMFNRAKLLNIGFQEALKDYDYNCFVFSDVDLIPMDDRNAYRCFSQPRHISVAMDKFGFSLPYVQYFGGVSALSKQQFLAINGFPNNYWGWGGEDDDIFNRLVHKGMSISRPNAVVGRCRMIRHSRDKKNEPNPQRFDRIAHTKETMRFDGLNSLTYKVLDVQRYPLYTQITVDIGTPR

SEQ ID NO: 5 - DNA, 2298 bp, Rattus norvegicus
cctctcgtgccccgctgatctctccagagtcccgcggctgggggtgtcgactttccgccacaggcggccggtgctgcgctaggtcctagccctccccccggcccccaccccccggcccctccttccatcgctgtggtcgggtagcgcctggcgggcggcctgcgggcgggccgtcctctcagccgtagcccaccccctcttaaagccgcggcgggaagatgaggtttcgtgagccgttcctgggcggcagcgccgcgatgccgggcgcgaccctgcagcgggcctgccgcctgctcgtggcggtctgcgcgctgcaccttggcgtcaccctggtctattacctctccggtcgcgatctgagccgcctgccccaactggtcggagtctcctcttcactgcaaggcggcacgaacggcgccgccgccagcaagcagccctcgggagagctccggccccggggcgcgcggccgccgcctcctttaggcgtctccccgaagcctcgcccgggttctgactccagccctgatgcggcttctggccccggcctgaagagcaacttgacttcggtgccaatgcccaccagcactggattgttgactctgcctgcttgccctgaggagtccccgctgctcgttggccccatggtgattgactttaatattcctgtggatctggagcttttggcaaagaagaacccagagataaagatgggcggccgttacttccccaaggactgtatctcccctcacaaggtggccatcattatcccattccgtaaccggcaggagcacctcaaatactggctgtattatttgcatccagtccttcagcgccagcaactcgactatggcatctacgtcatcaatcaggctggagacaccatgtttaatcgagctaagctgctcaacgttggctttcaagaggccttgaaagactatgactacaactgctttgtgttcagtgatgtggacctcattccaatggatgaccataatgcctacaggtgcttttcacagccacggcatatttctgtcgcaatggacaagttcgggtttagcctgccttacgttcagtattttggaggtgtctccgctctcagtaaacaacagttccttaccatcaatggatttcctaataattactggggctggggaggagaagatgatgacatttttaacagattagttcataaaggcatgtctatatcacgcccaaatgctgtggtaggcaggtgtcgcatgatccggcactcaagagacaagaaaaatgagcccaaccctcagaggtttgaccggatcgcacatacaaaggaaacgatgcgccttgatggtttgaactcacttacctaccaggtgttggacatacagagatacccgttatataccaaaatcacagtggacatcgggacaccaagatagcattttggtacaaataagagacccgaggatggccagagacctcagatgtgtgtctctgccagtcgactgggctggtccctctcatttgttcagtctgaatgatagttctccttccttaccattcagacatctttccagatgcccagggtgaatgtcacgtttacccacaacctggctcggcactgggtgaaattctacaaggtgtttatcagtgtaaaaacggtcagcctttggagaggtttcttggacacgtcacccccaaagagtcggaactgtgcccagctccaaccttagtgactgtgggtcatagtcccagtgctgaaactgctgaagtgtgacatgggtaagagctttgctttagtcggcgatgtccacacttcatgaccaatggaggctgctgctggattcggattcacaagatattcttgcatatttttttatacaaaaaatcaaaatttcaatcagtctcgtgttctgtccctttcctatgtcctctcaccggtatgcaactattacaatcactgcgtgtgcgtcttttcttagcaaaagagttttaaaacttgagcctggagcttggtgtcctgtgagtgtggctgccgaggccttgccctcagagcaggggccgggacactcacttccgtgacccgtctccagatccctgtctgatttctgaatgtaaagaggttttttgtggttgttgttttgttttgtttttgtttttagaagcagtttgtagtattttaaagaataaatcaagttttgattatgctataggttgatttttgtgttgatccaaattggaatagctattgagtgttttaagtcgtgactttatttttctgggcatgctatataaacttgaatttcctatgtaaaaaaa

SEQ ID NO: 6 - protein, 399 aa, Rattus norvegicus
MRFREPFLGGSAAMPGATLQRACRLLVAVCALHLGVTLVYYLSGRDLSRLPQLVGVSSSLQGGTNGAAASKQPSGELRPRGARPPPPLGVSPKPRPGSDSSPDAASGPGLKSNLTSVPMPTSTGLLTLPACPEESPLLVGPMVIDFNIPVDLELLAKKNPEIKMGGRYFPKDCISPHKVAIIIPFRNRQEHLKYWLYYLHPVLQRQQLDYGIYVINQAGDTMFNRAKLLNVGFQEALKDYDYNCFVFSDVDLIPMDDHNAYRCFSQPRHISVAMDKFGFSLPYVQYFGGVSALSKQQFLTINGFPNNYWGWGGEDDDIFNRLVHKGMSISRPNAVVGRCRMIRHSRDKKNEPNPQRFDRIAHTKETMRLDGLNSLTYQVLDIQRYPLYTKITVDIGTPR

SEQ ID NO: 7 - DNA, 20 bp, Artificial Sequence
  Synthetic Oligonucleotide
tattaaagtcaatcagcatg

SEQ ID NO: 8 - DNA, 20 bp, Artificial Sequence
  Synthetic Oligonucleotide
gggcggccgttactccccca

SEQ ID NO: 9 - DNA, 20 bp, Artificial Sequence
  Synthetic Oligonucleotide
atgatgatggccaccttgtg

SEQ ID NO: 10 - DNA, 20 bp, Artificial Sequence
  Synthetic Oligonucleotide
gagcgcaactccagtgcttg

SEQ ID NO: 11 - DNA, 23 bp, Artificial Sequence
  Synthetic Oligonucleotide
gaggacgtacctctgaggattgg

SEQ ID NO: 12 - DNA, 121 bp, Artificial Sequence
  oligonucleotide
atgctgtagtagggaggtgtcgaatgatccggcattcaagagacaagaaaaatgagcccaatcctcagaggtacgtcctctctgtgccttccctttatttatttatatgttagatttattt

SEQ ID NO: 13 - DNA, 121 bp, Artificial Sequence
  oligonucleotides
atgctgtagtagggaggtgtcgaatgatccggcattcaagagacaagaaaaatgagcccagtccccagaggtacgtcctctctgtgccttccctttatttatttatatgttagatttattt

(19) United States
(12) Patent Application Publication
(10) Pub. No.: US 2021/0274759 A1
(43) Pub. Date: Sep. 9, 2021

(54) A RODENT MODEL OF B4GALT1-MEDIATED FUNCTIONS

(71) Applicant: Regeneron Pharmaceuticals, Inc., Tarrytown, NY (US)

(72) Inventors: Giusy Della Gatta, Sleepy Hollow, NY (US); Qing Fang, Chappaqua, NY (US); Alan Shuldiner, Baltimore, MD (US)

(73) Assignee: Regeneron Pharmaceuticals, Inc., Tarrytown, NY (US)

(21) Appl. No.: 17/190,650

(22) Filed: Mar. 3, 2021

Related U.S. Application Data
(60) Provisional application No. 62/985,045, filed on Mar. 4, 2020.

(51) Int. Cl. A01K 67/027; C12N 15/11; C12N 9/22; C12N 9/10

(52) U.S. Cl. CPC A01K 67/0278; C12N 15/111; C12N 9/22; C12N 9/1048; A01K 2267/0362; A01K 2217/072; C12N 2310/20; A01K 2227/105; A01K 2207/05

(57) ABSTRACT

This disclosure relates to genetically modified animals. More specifically, this disclosure relates to rodent animals in which an endogenous B4galt1 gene has been modified, e.g., to introduce a mutation that encodes an Asn to Ser substitution in the encoded B4galt1 protein at a position corresponding to position 352 in a human B4GALT1 protein, or to introduce a loss of function mutation (e.g., in a select tissue such as the liver). This disclosure also relates to use of such rodent animals in elucidating the role of B4galt1 in lipid metabolism.

### CROSS REFERENCE TO RELATED APPLICATION

This application claims the benefit of priority from U.S. Provisional Application No. 62/985,045, filed Mar. 4, 2020, the entire contends of which are incorporated herein by reference.

### FIELD OF THE DISCLOSURE

This disclosure relates to genetically modified animals. More specifically, this disclosure relates to rodent animals in which an endogenous B4galt1 gene has been modified, e.g., to have a modified gene encoding a B4galt1 protein with reduced galactosyltransferase activity, to introduce a mutation that encodes an Asn to Ser substitution in the encoded B4galt1 protein at a position corresponding to position 352 in a human B4GALT1 protein, or to introduce a loss of function mutation (e.g., in a select tissue such as the liver). This disclosure also relates to use of such rodent animals in elucidating the role of B4galt1 in lipid metabolism.

### INCORPORATION BY REFERENCE OF SEQUENCE LISTING

The Sequence Listing in the ASCII text file, named as 37993_10700US01_SequenceListing of 27 KB, created on Feb. 24, 2021, and submitted to the United States Patent and Trademark Office via EFS-Web, is incorporated herein by reference.

### BACKGROUND ART

Various references, including patents, patent applications, accession numbers, technical articles, and scholarly articles are cited throughout the specification. Each reference is incorporated by reference herein, in its entirety and for all purposes.

Cardiovascular disease (CVD) accounts for 1 of every 3 deaths in the USA and is the leading cause of morbidity and mortality worldwide (Benjamin et al., Circulation, 2018. 137(12): p. e67-e492). Elevated low-density lipoprotein cholesterol (LDL-C) increases arterial plaque formation and atherosclerosis, and is a risk factor for coronary artery disease (CAD) (Nelson et al., Primary care, 2013. 40(1): p. 195-211). Deeper understanding of the genetic determinants of LDL-C may unveil novel targets for therapy that may be more efficacious and safer to treat or prevent CAD.

### SUMMARY OF THE DISCLOSURE

In one aspect, this disclosure provides genetically modified rodent animals (e.g., mice or rats) in which an endogenous B4galt1 gene has been modified.

In some embodiments, disclosed herein is a rodent that comprises a modification in an endogenous rodent β4 galactotransferase 1 (B4galt1) gene at an endogenous rodent B4galt1 locus.

In some embodiments, the modification in an endogenous rodent B4galt1 gene results in a modified rodent B4galt1 gene which encodes a B4galt1 protein with reduced galactosyltransferase activity. In some such embodiments, a modification comprises an addition, deletion, or substitution of one or more nucleotides in an endogenous rodent B4galt1 gene. In some embodiments, a modification results in or encodes a substitution of an amino acid in the B4galt1 protein such that the B4galt1 protein comprising the substitution displays reduced galactosyltransferase activity. In some such embodiments, the substitution is Asn to Ser at an amino acid position of a rodent B4galt1 protein corresponding to position 352 in a human B4GALT1 protein. In some embodiments, the modification is in the genome (i.e., germline genome) of the rodent. In some embodiments, the modification is introduced to the endogenous rodent B4galt1 gene in a target tissue or organ of the rodent.

In some embodiments, the modification in an endogenous rodent B4galt1 gene results in a modified rodent B4galt1 gene which encodes a B4galt1 protein comprising a substitution of Asn to Ser at an amino acid position corresponding to position 352 in a human B4GALT1 protein (also referred to as a rodent comprising “an N352S knock-in”). In some embodiments, the rodent is a mouse, and the substitution is at amino acid position 353 of a mouse B4galt1 protein. In some embodiments, the rodent is a rat and the substitution is at amino acid position 353 of a rat B4galt1 protein. In some embodiments, the modification is in the genome (i.e., germline genome) of the rodent. In some embodiments, the rodent is heterozygous for the modification. In some embodiments, the rodent is homozygous for the modification. A rodent comprising an N352S knock-in displays a decreased level of LDL-C, as compared to a wild type rodent without the modification. In some embodiments, the modification is introduced to the endogenous rodent B4galt1 gene in a target tissue or organ of the rodent.

In some embodiments, the modification in an endogenous rodent B4galt1 gene is a loss of function mutation. In some embodiments, the loss of function mutation comprises an insertion, deletion or substitution of one or more nucleotides which results in, in some embodiments, a deletion, in whole or in part, of the coding sequence of the endogenous rodent B4galt1 gene. In some embodiments, the insertion, deletion or substitution of one or more nucleotides occurs in exon 2 of the endogenous rodent B4galt1 gene. In some embodiments, the loss of function mutation is in the genome (e.g., germline genome) of the rodent. In some embodiments, the modification is introduced to the endogenous rodent B4galt1 gene in a target tissue or organ of the rodent. In some embodiments, the target organ is the liver.

In a further aspect, provided herein is an isolated rodent (e.g., mouse or rat) cell or tissue that comprises a modification described herein, i.e., a modification in an endogenous rodent B4galt1 gene at an endogenous rodent B4galt1 locus. In some embodiments, the cell or tissue can be isolated from a rodent comprising the modification. In some embodiments, an isolated rodent cell is a rodent embryonic stem cell.

In some embodiments, the modification in an endogenous rodent B4galt1 gene results in a modified rodent B4galt1 gene which encodes a B4galt1 protein with reduced galactosyltransferase activity. In some such embodiments, a modification comprises an addition, deletion, or substitution of one or more nucleotides in an endogenous rodent B4galt1 gene. In some embodiments, a modification results in or encodes a substitution of an amino acid in the B4galt1 protein such that the B4galt1 protein comprising the substitution displays reduced galactosyltransferase activity. In some such embodiments, the substitution is Asn to Ser at an amino acid position of a rodent B4galt1 protein corresponding to position 352 in a human B4GALT1 protein.

In some embodiments of an isolated rodent cell or tissue, the modification in an endogenous rodent B4galt1 gene results in a modified rodent B4galt1 gene which encodes a B4galt1 protein comprising a substitution of Asn to Ser at an amino acid position corresponding to position 352 in a human B4GALT1 protein (also referred to as a rodent cell or tissue comprising “an N352S knock-in”). In some embodiments, the rodent cell or tissue is a mouse cell or tissue, and the substitution is at amino acid position 353 of a mouse B4galt1 protein. In some embodiments, the rodent is a rat cell or tissue and the substitution is at amino acid position 353 of a rat B4galt1 protein. In some embodiments, the rodent cell or tissue is heterozygous for the modification. In some embodiments, the rodent cell or tissue is homozygous for the modification. In some embodiments, the rodent cell or tissue is a liver cell or tissue.

In some embodiments of an isolated rodent cell or tissue, the modification in an endogenous rodent B4galt1 gene is a loss of function mutation. In some embodiments, the loss of function mutation comprises an insertion, deletion or substitution of one or more nucleotides which results in, in some embodiments, a deletion, in whole or in part, of the coding sequence of the endogenous rodent B4galt1 gene. In some embodiments, the insertion, deletion or substitution of one or more nucleotides occurs in exon 2 of the endogenous rodent B4galt1 gene.

Also provided herein is a rodent (e.g., mouse or rat) embryo that comprises a rodent embryonic cell disclosed herein.

In a further aspect, a method of making a genetically modified rodent and a rodent made by the method are provided.

In some embodiments, the method comprising (i) introducing a modification into an endogenous rodent B4galt1 gene at an endogenous rodent B4galt1 locus of a rodent embryonic stem (ES) cell, thereby obtaining a modified rodent ES cell comprising a modified rodent B4galt1 gene; and (ii) making the genetically modified rodent using the modified rodent ES cell.

In some embodiments, the modification in an endogenous rodent B4galt1 gene results in a modified rodent B4galt1 gene which encodes a B4galt1 protein with reduced galactosyltransferase activity. In some such embodiments, a modification comprises an addition, deletion, or substitution of one or more nucleotides in an endogenous rodent B4galt1 gene. In some embodiments, a modification results in or encodes a substitution of an amino acid in the B4galt1 protein such that the B4galt1 protein comprising the substitution displays reduced galactosyltransferase activity. In some such embodiments, the substitution is Asn to Ser at an amino acid position of a rodent B4galt1 protein corresponding to position 352 in a human B4GALT1 protein.

In some embodiments, the modification in an endogenous rodent B4galt1 gene results in a modified rodent B4galt1 gene which encodes a B4galt1 protein comprising a substitution of Asn to Ser at an amino acid position corresponding to position 352 in a human B4GALT1 protein. In some embodiments, the rodent is a mouse and the substitution is at amino acid position 353 of a mouse B4galt1 protein. In some embodiments, the rodent is a rat and the substitution is at amino acid position 353 of a rat B4galt1 protein.

In some embodiments, the modification in an endogenous rodent B4galt1 gene is a loss of function mutation. In some embodiments, the loss of function mutation comprises an insertion, deletion or substitution of one or more nucleotides which results in, in some embodiments, a deletion, in whole or in part, of the coding sequence of the endogenous rodent B4GalT-1 gene. In some embodiments, the insertion, deletion or substitution of one or more nucleotides occurs in exon 2 of the endogenous rodent B4(GalT-1 gene.

In some embodiments, the modification is introduced into the endogenous rodent B4galt1 gene in a rodent ES cell through a gene editing system. In some embodiments, the gene editing system is a CRISPR/Cas9 system. In some embodiments, the gene editing system comprises a guide RNA, a Cas9 enzyme, and a single stranded oligodeoxynucleic acid molecule (ssODN). In some embodiments, a guide RNA and ssODN are introduced (e.g., via transfection or electroporation) into a rodent ES cell, wherein the rodent ES cell already expresses, or has been modified to express, a Cas9 enzyme.

In some embodiments, a method of making a genetically modified rodent comprises introducing a modification into an endogenous rodent B4galt1 gene at an endogenous rodent B4galt1 locus in a target tissue of a rodent, thereby obtaining the genetically modified rodent.

In some embodiments, the modification in an endogenous rodent B4galt1 gene results in a modified rodent B4galt1 gene which encodes a B4galt1 protein with reduced galactosyltransferase activity. In some such embodiments, a modification comprises an addition, deletion, or substitution of one or more nucleotides in an endogenous rodent B4galt1 gene. In some embodiments, a modification results in or encodes a substitution of an amino acid in the B4galt1 protein such that the B4galt1 protein comprising the substitution displays reduced galactosyltransferase activity. In some such embodiments, the substitution is Asn to Ser at an amino acid position of a rodent B4galt1 protein corresponding to position 352 in a human B4GALT1 protein.

In some such embodiments, the modification in an endogenous rodent B4galt1 gene results in a modified rodent B4galt1 gene which encodes a B4galt1 protein comprising a substitution of Asn to Ser at an amino acid position corresponding to position 352 in a human B4GALT1 protein. In some embodiments, the rodent is a mouse and the substitution is at amino acid position 353 of a mouse B4galt1 protein. In some embodiments, the rodent is a rat and the substitution is at amino acid position 353 of a rat B4galt1 protein.

In some embodiments, the modification in an endogenous rodent B4galt1 gene is a loss of function mutation. In some embodiments, the loss of function mutation comprises an insertion, deletion or substitution of one or more nucleotides which results in, in some embodiments, a deletion, in whole or in part, of the coding sequence of the endogenous rodent B4galt1 gene. In some embodiments, the insertion, deletion or substitution of one or more nucleotides occurs in exon 2 of the endogenous rodent B4galt1 gene.

In some embodiments, the modification is introduced into the endogenous rodent B4galt1 gene through a gene editing system. In some embodiments, the gene editing system is a CRISPR/Cas9 system. In some embodiments, a guide RNA of the CRISPR/Cas9 system is delivered into the rodent by an AAV system. In some embodiments, the AAV system targets delivery of the guide RNA into the liver of the rodent. In some embodiments, the Cas9 enzyme is expressed in the rodent prior to introduction of the guide RNA into the rodent.

In another aspect, provided herein is a method of breeding rodents and rodent progenies obtained.

In some embodiments, disclosed herein is a method comprising breeding a first rodent whose genome comprises a modification in an endogenous rodent B4galt1 gene (i.e., comprises a modified rodent B4galt1 gene), with a second rodent, resulting in a progeny rodent whose genome comprises the modification in the rodent B4galt1 gene.

In some embodiments, disclosed herein is a progeny obtained from breeding a first rodent whose genome comprises a modification in an endogenous rodent B4galt1 gene, with a second rodent, wherein the genome of the progeny comprises the modification in the rodent B4galt1 gene.

In a further aspect, provided herein is a method of testing the effect of a compound on B4galt1 and lipid metabolism, the method comprising (i) providing a rodent comprising a modification in an endogenous rodent B4galt1 gene as described herein, and providing a wild type rodent without the modification, (ii) administering a candidate B4galt1 inhibiting compound to the wild type rodent; (iii) examining the rodent with the modification and the wild type rodent to measure serum LDL-C levels; and (iv) comparing the measurements from the wild type rodent administered with the compound, from the wild type rodent before the administration of the compound, and from the rodent with the modification to determine whether the candidate compound inhibits the activity of B4galt1.

### BRIEF DESCRIPTION OF THE DRAWINGS

FIG. 1A depicts an alignment of human B4GALT1 (SEQ ID NO: 2) and mouse B4galt1 (SEQ ID NO: 4) protein sequences. The amino acids common to both sequences are boxed. The Asn residue at position 352 of the human protein and the corresponding Asn residue at position 353 of the mouse protein are shown enclosed in a box.

FIG. 1B depicts mouse B4galt1 gene (top) and protein (bottom). Top: the horizontal line/bar represents the mouse B4galt1 gene locus, with exons shown by the vertical bars above the line. The open, unfilled portions of exon 1 and exon 6 represent the 5′ untranslated region (5′ UTR) and the 3′ UTR, respectively. Bottom: the horizontal bar represents the mouse B4galt1 protein. The junctions between exons are shown by vertical lines within the bar, and the amino acid positions corresponding to the junctions are shown below the vertical lines. The N353S substitution is shown by an asterisk.

FIG. 1C depicts an exemplary strategy for introducing a mutation into exon 5 of a mouse B4galt1 gene resulting in an N353S substitution in the mouse B4galt1 protein. A guide RNA sequence (SEQ ID NO: 11), which is complementary to a portion of the wild type exon 5 sequence (SEQ ID NO: 12), directs a nuclease (e.g., Cas9) to introduce a double-stranded break. Upon repair using as template a single stranded donor oligodeoxynucleotide (ssODN) sequence (SEQ ID NO: 13), a mutation (a nucleotide substitution of A to G) is introduced resulting in an N353S substitution in the encoded B4galt1 protein.

FIGS. 2A-2B demonstrate the effects of B4galt1 N352S Knock-In on lipid and enzyme levels in plasma of female (2A) and male (2B) mice.

FIGS. 3A-3E demonstrate the effects of liver specific B4galt1 ablation on lipid and enzyme levels in plasma. 3A, 3B: Percentage of editing of b4galt1 in liver and spleen at 14 weeks from viral transduction by AAV8. In each experimental group, the number of reads containing B4galt1 INDELs was compared to the number of reads with B4galt1 wild type sequence (as described in the Methods section in Example 2). 3C: Analysis of B4galt1 mRNA levels in liver by Taqman at 14 weeks from viral transduction by AAV8. Expression of B4galt1 was calculated relative to Gapdh housekeeping gene. Values represent the mean of 4 technical replicates per condition. 3D, 3E: Shown are plasma levels for LDL-C and AST from B4galt1 liver-knockout and Cfb knockout control. After the two weeks time point, a periodic bleeding was performed every 4 weeks for a total study period of 12 weeks from the injection of viral vector. Values represent the mean of 3-5 biological replicates. Error bars show standard error.

FIGS. 4A-4J demonstrate the effects of liver specific B4galt1 ablation on lipid and enzyme levels in plasma. 4A, 4B: Percentage of editing of b4galt1 in liver and spleen at 14 weeks from viral transduction by AAV8 carrying two different gRNA designed against exon 2 of B4galt1. In each experimental group, the number of reads containing b4galt1 INDELs was compared to the number of reads with b4galt1 wild type sequence (see Methods). 4C: Analysis of b4galt1 mRNA levels in liver by Taqman at 14 weeks from viral transduction by AAV8. Expression of b4galt1 was calculated relative to gapdh housekeeping gene. Values represent the mean of 4 technical replicates per condition. 4D-4J: Shown are the plasma levels for: LDL-C; AST; T-cholesterol: HDL-C; NEFA: Triglycerides and ALT measured in b4galt1 liver specific knockout and cfb knockout control. After the two weeks time point, a periodic bleeding was performed every 4 weeks for a total study period of 12 weeks from the injection of viral vector. Values represent the mean of 3-5 biological replicates. Error bars show standard error.

### DETAILED DESCRIPTION

Disclosed herein are genetically modified rodents suitable for use as an animal model of human metabolisms (e.g., lipid metabolism) and diseases. In particular, disclosed herein are rodent animals in which an endogenous B4galt1 gene has been modified, e.g., to encode a B4galt1 protein with reduced galactosyltransferase activity, to introduce a mutation resulting in an amino acid substitution, or to introduce a loss of function mutation. Also disclosed herein is use of such rodent animals in elucidating the role of B4galt1 in lipid metabolism.

### B4GALT1

B4GALT1 (or B4galt1 from non-human sources) is a member of the beta-1,4-galactosyltransferase gene family which encode type II membrane-bound glycoprotein that plays a critical role in the processing of N-linked oligosaccharide moieties in glycoproteins. Impairment of B4GALT1 (or B4galt1) activity has the potential to alter the structure of N-linked oligosaccharides and introduce aberrations in glycan structure that have the potential to alter glycoprotein function.

Human 4GALT1 gene is located at 9p21.1 on chromosome 9, is about 56 kb in length with 6 exons, and encodes a polypeptide of 398 amino acids. Mouse B4galt1 gene is located on chromosome 4, is about 49 kb in length with 6 exons, and encodes a protein of 399 amino acids. B4GALT1 is highly conserved across species. Exemplary mRNA and protein sequences from human, mouse and rat are available in GenBank under the accession numbers listed in Table 1, and are also set forth as SEQ ID NOS: 1-6 in the Sequence Listing.

TABLE 1

| SEQ | | |
|---|---|---|
| ID NO | Description | Features |
| 1 | Homo sapiens B4GALT1 | Length: 4214 bp |
| | mRNA, NM_001497.3 | CDS: nt. 190-1386 |
| | | Exons 1-6: nt. 1-601, 602-837, |
| | | 838-1025, 1026-1148, 1149-1252, |
| | | 1254-4199. |
| | | PolyA site: nt. 4199. |
| 2 | Homo sapiens B4GALT1 | Length: 398 aa |
| | protein, NP_001488.2 | Transmembrane: aa 25-44: |
| | | Mature, soluble form: aa 78-398 |
| 3 | Mus musculus B4galt1 | Length: 4535 bp |
| | mRNA, NM_022305.4 | CDS: nt 733-1932 |
| | | Exons 1-6: nt. 1-1147, 1148-1383, |
| | | 1384-1571, 1572-1694, 1695-1799, |
| | | 1800-4535 |
| | | PolyA signal sequence: nt. |
| | | 4493-4498 |
| | | PolyA site: nt. 4515 |
| 4 | Mus musculus B4galt1 | Length: 399 aa |
| | protein, NP_071641.1 | Transmembrane: aa 25-44: |
| 5 | Rattus norvegicus B4galt1 | Length: 2298 bp |
| | mRNA, NM_0153287.1 | CDS: nt. 219-1418 |
| | | Exons 1-6: nt. 1-633, 634-869, |
| | | 870-1057, 1058-1180, 1181-1285, |
| | | and 1286-2292. |
| 6 | Rattus norvegicus B4galt1 | Length: 399 aa |
| | protein, NP_445739.1 | |

### Rodents Comprising a Modified B4galt1 Gene

This disclosure provides rodents (e.g., mice and rats) in which an endogenous B4galt1 gene has been modified, e.g., to introduce a mutation resulting in an amino acid substitution (e.g., a substitution that reduces the activity of the B4galt1 protein), or to introduce a loss of function mutation.

The term “mutation” includes an addition, deletion, or substitution of one or more nucleotides in a gene. As used herein, the terms “mutation”, “alteration”, and “modification” are used interchangeably. A mutant gene (or a mutant allele of a gene) is understood herein to include a mutation, alteration or modification relative to a wild type gene or a reference gene. In some embodiments, a mutation is a substitution of a single nucleotide. In some embodiments, a mutation is a deletion of one or more nucleotides, e.g., one or more nucleotides in the coding sequence of a gene. In some embodiments, a mutation in a gene includes a deletion of a contiguous nucleic acid sequence, e.g., one or more exons, of a gene. In some embodiments, a mutation in a gene results in an addition, deletion, or substitution of one or more amino acids in the encoded protein. In some embodiments, a mutation in a gene does not change the encoded amino acid.

The term “loss of function” includes a complete loss of function and a partial loss of function. In some embodiments, a modification or alteration in a gene results in expression of a polypeptide with at least diminished functionality and, in some cases, with a substantially diminished functionality or complete lack of functionality relative to a polypeptide encoded by a reference gene not having the modification or alteration. Thus, a genetic modification may cause a complete loss of function or a partial loss of function. In some embodiments, disclosed herein is a rodent animal which comprises a modification in an endogenous rodent B4galt1 gene wherein the rodent B4galt1 gene comprising the modification encodes a B4galt1 protein with reduced galactosyltransferase activity. In some embodiments, a modification comprises an addition, deletion, or substitution of one or more nucleotides in an endogenous rodent B4galt1 gene. In some embodiments, a modification results in or encodes a substitution of an amino acid in the B4galt1 protein. In some embodiments, the substitution is Asn to Ser at an amino acid position of a rodent B4galt1 protein corresponding to position 352 in a human B4GALT1 protein. In some embodiments, a reduction in galactosyltransferase activity may be, for example, inhibition or diminishment of activity relative to a native or wild type rodent B4galt1 protein encoded by a wild type rodent B4galt1 gene (a rodent B4galt1 gene without the modification).

In some embodiments, disclosed herein is a rodent animal comprising a modification in an endogenous rodent B4galt1 gene such that the modified rodent B4galt1 gene encodes a modified B4galt1 protein comprising an Asn to Ser substitution at an amino acid position corresponding to position 352 in a human B4galt1 protein. Such a rodent is also referred to herein as a rodent having a N352S knock-in. The modification can be, for example, a substitution of a nucleotide in the codon for Asn at a position corresponding to position 352 in a human B4galt1 protein, resulting in an Asn to Ser substitution in the encoded rodent B4galt1 protein. Such a modification is also said to “encode an N to S substitution”. The N352S variation in human B4GALT1 is believed to associate with decreased LDL-C.

As used herein, the phrase “corresponding to” or grammatical variations thereof when used in the context of the numbering of positions in a given polypeptide or nucleic acid molecule refers to the numbering of a specified reference polypeptide or nucleic acid molecule when the given amino acid or nucleic acid molecule is compared to the reference molecule (e.g., with the reference molecule herein being a wild type human B4GALT1 polypeptide or a wild type human B4GALT1 nucleic acid molecule). In other words, the position of an amino acid residue or nucleotide in a given polymer is designated with respect to the reference molecule rather than by the actual numerical position of the amino acid residue or nucleotide within the given polymer. For example, a given amino acid sequence can be aligned to a reference sequence by introducing gaps to optimize residue matches between the two sequences. In these cases, although the gaps are present, the numbering of the residue in the given amino acid or nucleic acid sequence is made with respect to the reference sequence to which it has been aligned.

For example, a position within a rodent B4galt1 protein that corresponds to position 352 of a human B4GALT1 protein can easily be identified by performing a sequence alignment between the rodent B4galt1 protein and the amino acid sequence of the human B4GALT1 protein (e.g., SEQ ID NO: 2). A variety of computational algorithms exist that can be used for performing a sequence alignment in order to identify an amino acid position that corresponds to position 352 in SEQ ID NO: 2. For example, by using the NCBI BLAST algorithm (Altschul et al. 1997 Nucleic Acids Res. 25: 3389-3402) or CLUSTALW software (Sievers and Higgins 2014 Methods Mol. Biol. 1079: 105-116.) sequence alignments may be performed. However, sequences can also be aligned manually.

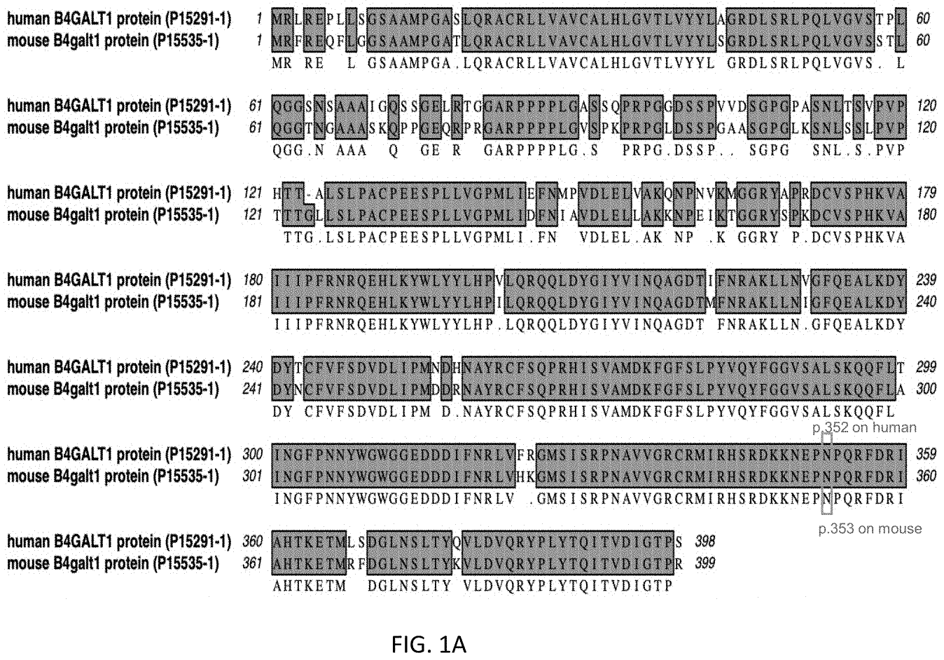

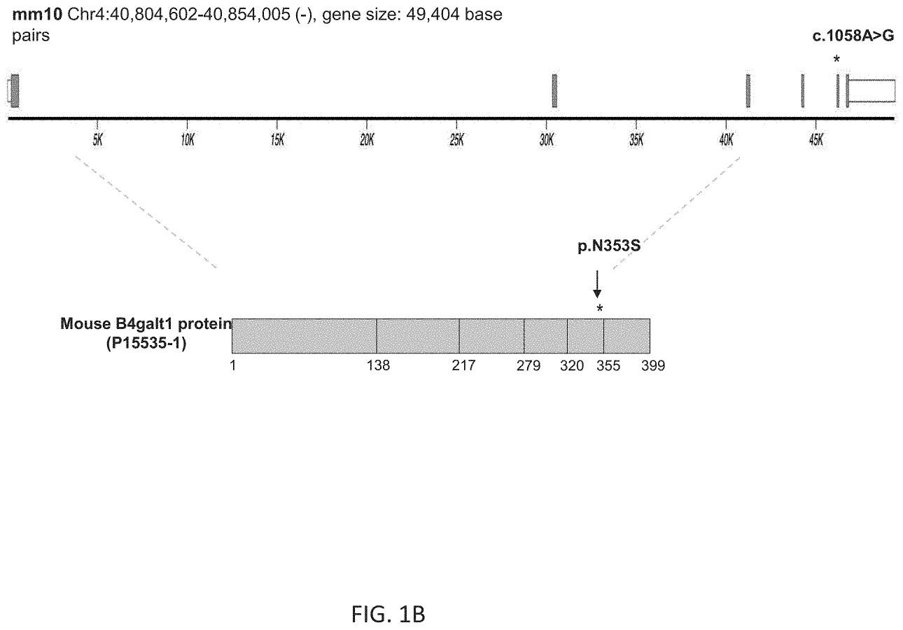

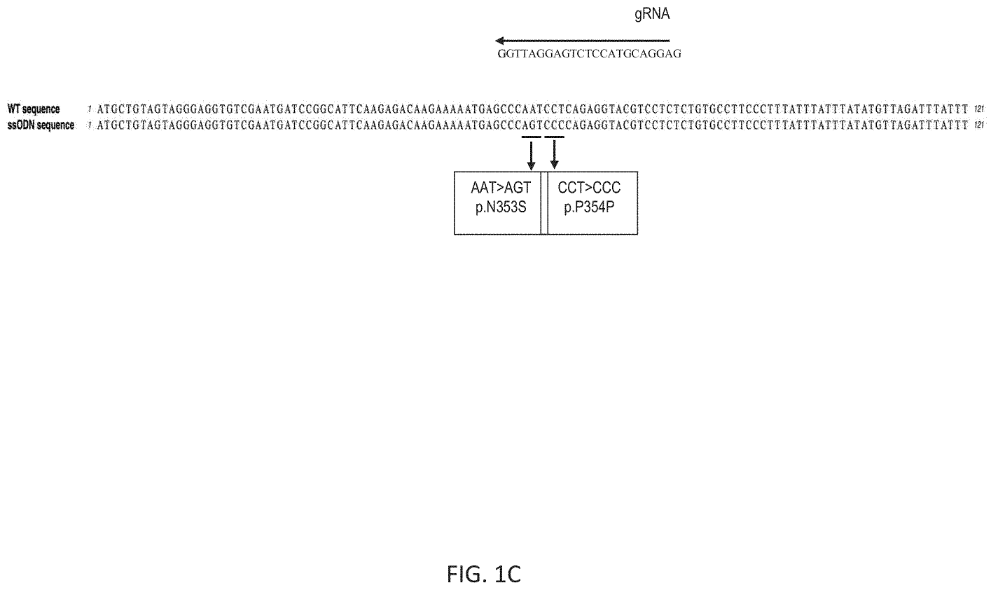

In some embodiments, the rodent is a mouse comprising a modification in an endogenous mouse B4galt1 gene such that the modified mouse B4galt1 gene encodes a modified mouse B4galt1 protein comprising an Asn to Ser substitution at an amino acid position corresponding to position 352 in a human B4galt1 protein. Position 353 in a mouse B4galt1 protein (e.g., SEQ ID NO: 4) corresponds to position 352 of a human B4GALT1 protein such as SEQ ID NO: 2. See, for example, FIG. 1A.

In some embodiments, the rodent is a rat comprising a modification in an endogenous rat B4galt1 gene such that the modified rat B4galt1 gene encodes a modified rat B4galt1 protein comprising an Asn to Ser substitution at an amino acid position corresponding to position 352 in a human B4galt1 protein. Position 353 in a rat B4galt1 protein (e.g., SEQ ID NO: 6) corresponds to position 352 of a human B4GALT1 protein such as SEQ ID NO: 2.

In some embodiments, disclosed herein is a rodent animal which comprises a modification in an endogenous rodent B4galt1 gene wherein the modification is a loss of function mutation.

In some embodiments, a loss of function mutation in a rodent B4galt1 gene includes a deletion of at least a portion of an endogenous rodent B4galt1 gene.

A “portion” of a gene is used herein interchangeably with a “fragment” of a gene, which includes references to contiguous nucleotide sequence portions of a gene, including, for example, a 5′ regulatory region (e.g., promoter), a 5′ non-coding exonic sequence, a 3′ non-coding exonic sequence, a 5′ or 3′ untranslated region (UTR), an exon in full or in part, an intron in full or in part, a 3′ region downstream of the last exon, or combinations thereof. In some embodiments, a portion of a gene refers to the coding region of the gene, e.g., a nucleic acid (genomic DNA or cDNA) comprising the ATG start codon through the stop codon of the gene.

In some embodiments, a modification in an endogenous rodent B4galt1 gene includes a loss of function mutation that results from an insertion, deletion or substitution of one or more nucleotides (e.g., in an exon), leading to a deletion of at least a portion of the coding sequence.

In some embodiments, a modification in an endogenous rodent B4galt1 gene (e.g., a point mutation resulting in an N to S substitution at a position corresponding to 352 in human B4GALT1, or a loss of function mutation), is in the genome (i.e., germline genome) of a rodent animal. In some embodiments, a rodent is heterozygous for a modification. In some embodiments, a rodent is homozygous for a modification.

In some embodiments, a modification in an endogenous rodent B4galt1 gene is present in a selected or targeted tissue or organ of a rodent, i.e., a tissue or organ-specific modification of an endogenous rodent B4galt1 gene. In some embodiments, a modification in an endogenous rodent B4galt1 gene is present in the liver of a rodent. In some embodiments, a loss of function mutation in an endogenous rodent B4galt1 gene is present in the liver of a rodent to achieve liver-specific ablation of a rodent B4galt1 gene.

In some embodiments, a rodent animal disclosed herein is incapable of expressing a wild type rodent B4galt1 protein. For example, a rodent is provided where one copy of the endogenous rodent B4galt1 gene contains a modification (e.g., a modification resulting in a substitution corresponding to N352S in human B4GALT1) and the other copy is disrupted or deleted. Alternatively, the rodent animal is homozygous for the modification and is consequently incapable of expressing a wild type rodent B4galt1 protein.

Rodent animals provided herein, as a result of modification in an endogenous B4galt1 gene (either homozygous or heterozygous for an N352S knock-in, or a liver-specific ablation), exhibit decreased levels of LDL-C, e.g., by at least 10%, at least 15%, at least 20%, at least 25%, at least 30%, at least 40%, at least 45%, at least 50%, at least 60%, at least 70%, at least 80%, at least 90% or more, as compared to wild type mice (i.e., mice without the modification).

For any of the embodiments described herein, the rodents can include, for example, mice, rats, and hamsters.

In some embodiments, the rodent is a mouse. In some embodiments, the rodent is a mouse of a C57BL strain, for example, a C57BL strain selected from C57BL/A, C57BL/An, C57BL/GrFa, C57BL/KaLwN, C57BL/6, C57BL/6J, C57BL/6ByJ, C57BL/6NJ, C57B10, C57BL/10ScSn, C57BL/10Cr, and C57BL/Ola. In other embodiments, the rodent is a mouse of a 129 strain, for example, a 129 strain selected from the group consisting of 129P1, 129P2, 129P3, 129X1, 129S1 (e.g., 129S1/SV, 129S1/SvIm), 129S2, 129S4, 12955, 129S9/SvEvH, 129/SvJae, 129S6 (129/SvEvTac), 129S7, 129S8, 129T1, 129T2 (see, e.g., Festing et al. (1999), Mammalian Genome 10:836: Auerbach et al. (2000), Biotechniques 29(5):1024-1028, 1030, 1032). In some embodiments, the rodent is a mouse that is a mix of an aforementioned 129 strain and an aforementioned C57BL/6 strain. In certain embodiments, the mouse is a mix (i.e., hybrid) of aforementioned 129 strains, or a mix of aforementioned C57BL strains, or a mix of a C57BL strain and a 129 strain. In certain embodiments, the mouse is a mix of a C57BL/6 strain with a 129 strain. In specific embodiments, the mouse is a VGF1 strain, also known as F1H4, which is a hybrid of C57BL/6 and 129. In other embodiments, the mouse is a BALB strain, e.g., BALB/c strain. In some embodiments, the mouse is a mix of a BALB strain and another aforementioned strain.

In some embodiments, the rodent is a rat. In certain embodiments, the rat is selected from a Wistar rat, an LEA strain, a Sprague Dawley strain, a Fischer strain, F344, F6, and Dark Agouti. In other embodiments, the rat is a mix of two or more strains selected from the group consisting of Wistar, LEA, Sprague Dawley, Fischer, F344, F6, and Dark Agouti.

### Methods of Making a Rodent Comprising a Modification in a B4galt1 Gene

The rodents provided herein, which comprises a modification in an endogenous B4galt1 gene, can be made using a variety of methods.

In some embodiments, a modification can be introduced into an endogenous rodent B4galt1 gene using a gene editing system (also known as a “targeted genome editing” system).

In some embodiments, the gene editing system is selected from CRISPR/Cas system, zinc finger nucleases (ZFNs), and TAL effector nucleases (TALENs). ZFNs are reviewed in Carroll, D. (Genetics, 188.4 (2011): 773-782), and TALENs are reviewed in Zhang et al. (Plant Physiology, 161.1 (2013): 20-27), which are incorporated herein in their entirety.

In some embodiments, the CRISPR/Cas system is used to introduce a modification into an endogenous rodent B4galt1 gene. The CRISPR/Cas system is a method based on the bacterial type II CRISPR (clustered regularly interspaced short palindromic repeats)/Cas (CRISPR-associated) immune system. The CRISPR/Cas system allows targeted cleavage of genomic DNA guided by a customizable small noncoding RNA (guide RNA or gRNA), resulting in gene modifications by both non-homologous end joining (NHEJ) and homology-directed repair (HDR) mechanisms. CRISPR-Cas and similar gene editing systems are known in the art with reagents and protocols readily available. Exemplary genome editing protocols are described in Jennifer Doudna, and Prashant Mali, “CRISPR-Cas: A Laboratory Manual” (2016) (CSHL Press, ISBN: 978-1-621821-30-4). and Ran, F. Ann, et al. (Nature Protocols (2013), 8 (11): 2281-2308), which are incorporated herein in their entirety.

In some embodiments, a modification is introduced into an endogenous rodent B4galt1 gene using the CRISPR/Cas system and an exogenous donor nucleic acid. In some embodiments, rodent ES cells are used and express, or are modified to express, a Cas nuclease. In some embodiments, the Cas protein is selected from the group consisting of Cas1, Cas1B, Cas2, Cas3, Cas4, Cas5, Cas5e (aka. CasD), Cas6, Cas6e, Cas6f, Cas7, Cas8a1, Cas8a2, Cas8b, Cas8c, Cas9 (aka. Csn1 or Csx12), Cas10, Cas10d, CasF, CasG, CasH, Csy1, Csy2, Csy3, Cse1 (aka. CasA), Cse2 (aka. CasB), Cse3 (aka. CasE), Cse4 (aka. CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In a specific embodiment, the Cas nuclease is Cas9.

In some embodiments, an exogenous donor nucleic acid comprises a deoxyribonucleic acid (DNA). In some embodiments, an exogenous donor nucleic acid comprises a ribonucleic acid (RNA). In some embodiments, an exogenous donor nucleic acid is single-stranded. In some embodiments, an exogenous donor nucleic acid is double-stranded. In some embodiments, an exogenous donor nucleic acid is in linear form. In some embodiments, an exogenous donor nucleic acid is in circular form. In some embodiments, an exogenous donor nucleic acid is a single-stranded oligodeoxynucleotide (ssODN). See, e.g., Yoshimi et al. (2016) Nat. Commun. 7:10431, US Patent Publication Nos. 2019/0032155 and 2019/0032156, all of which are incorporated by reference in their entireties.

In some embodiments, an exogenous donor nucleic acid is between about 50 nucleotides to about 5 kb in length, is between about 50 nucleotides to about 3 kb in length, or is between about 50 to about 1,000 nucleotides in length. In some embodiments, an exogenous donor nucleic acid is between about 40 to about 200 nucleotides in length. For example, an exogenous donor nucleic acid can be between about 50-60, 60-70, 70-80, 80-90, 90-100, 100-110, 110-120, 120-130, 130-140, 140-150, 150-160, 160-170, 170-180, 180-190, or 190-200 nucleotides in length. In some embodiments, an exogenous donor nucleic acid is between about 50-100, 100-200, 200-300, 300-400, 400-500, 500-600, 600-700, 700-800, 800-900, or 900-1000 nucleotides in length. In some embodiments, an exogenous donor nucleic acid is between about 1-1.5, 1.5-2, 2-2.5, 2.5-3, 3-3.5, 3.5-4, 4-4.5, or 4.5-5 kb in length. In some embodiments, an exogenous donor nucleic acid is no more than 5 kb, 4.5 kb, 4 kb, 3.5 kb, 3 kb, 2.5 kb, 2 kb, 1.5 kb, 1 kb, 900 nucleotides, 800 nucleotides, 700 nucleotides, 600 nucleotides, 500 nucleotides, 400 nucleotides, 300 nucleotides, 200 nucleotides, 100 nucleotides, or 50 nucleotides in length.

In some embodiments, an exogenous donor nucleic acid is an ssODN that is between about 80 nucleotides and about 200 nucleotides in length. In some embodiments, an exogenous donor nucleic acid is an ssODN that is between about 80 nucleotides and about 3 kb in length. In some embodiments, an ssODN has homology arms that are each between about 40 nucleotides and about 60 nucleotides in length. In some embodiments, an ssODN has homology arms that are each between about 30 nucleotides and 100 nucleotides in length. In some embodiments, the homology arms are symmetrical having the same number of nucleotides in each homology arm. In some embodiments, the homology arms are asymmetrical having different numbers of nucleotides in each homology arm.

In some embodiments, an exogenous donor nucleic acid is designed to delete a nucleic acid sequence of interest at a target genomic locus and replace it with a nucleic acid insert. In some embodiments, an exogenous donor nucleic acid is designed to introduce a substitution of one or more nucleotides. An example of an exogenous donor nucleic acid is ssODN having the nucleotide sequence of SEQ ID NO. 13.

In some embodiments, the CRISPR/Cas system and an exogenous donor nucleic acid are introduced into rodent embryonic stem (ES) cells in order to introduce a modification in an endogenous rodent B4galt1 gene in the rodent ES cells. In some embodiments, the rodent ES cells already express some components of the CRISPR/Cas system. In a specific embodiment, the rodent ES cell is a Cas-ready mouse embryonic cell described in US 2019/0032155 A1 (Regeneron Pharmaceuticals, Inc.), which is incorporated herein in its entirety.

In some embodiments, guide RNAs, Cas proteins, and/or exogenous donor nucleic acids are introduced into a cell or a non-human animal (e.g., a rodent) via any delivery method (e.g., Adeno-associated virus (AAV), lipid nanoparticle (LNP), or hydrodynamic gene delivery (HDD)) and any route of administration.

In some embodiments, gene editing components (e.g., guide RNA, Cas proteins, and/or exogenous donor nucleic acids) are delivered via AAV-mediated delivery. See, e.g., US Patent Publication No. 2016/0159436, herein incorporated by reference in its entirety. Multiple serotypes of AAV have been identified. These serotypes differ in the types of cells they infect (i.e., their tropism), allowing preferential transduction of specific cell types. Serotypes for CNS tissue include AAV1, AAV2, AAV4, AAV5, AAV8, and AAV9. Serotypes for heart tissue include AAV1, AAV8, and AAV9. Serotypes for kidney tissue include AAV2. Serotypes for lung tissue include AAV4, AAV5, AAV6, and AAV9. Serotypes for pancreas tissue include AAV8. Serotypes for photoreceptor cells include AAV2, AAV5, and AAV8. Serotypes for retinal pigment epithelium tissue include AAV1, AAV2, AAV4, AAV5, and AAV8. Serotypes for skeletal muscle tissue include AAV1, AAV6, AAV7, AAV8, and AAV9. Serotypes for liver tissue include AAV7, AAV8, and AAV9, and particularly AAV8.

In some embodiments, tropism of the AAV is further refined through pseudotyping, which is the mixing of a capsid and a genome from different viral serotypes. For example, AAV2/8 indicates a virus containing the genome of serotype 2 packaged in the capsid from serotype 8. In some embodiments, pseudotyped viruses display improved transduction efficiency, as well as altered tropism. In some embodiments, hybrid capsids derived from different serotypes are used to alter viral tropism.

In some embodiments, the endogenous rodent B4galt1 gene in the liver is targeted for modification by using the CRISPR/Cas system and an AAV system.

In some embodiments, the liver specific targeting is achieved by an AAV system with tropism towards the liver. In a specific embodiment, the AAV system is selected from AAV8, AAV2/8, AAV7, AAV9, and a hybrid AAV strain comprising hybrid capsids derived from different serotypes with liver tropism (e.g., any combination of capsids from liver tropic AAVs: AAV7, AAV8 and AAV9).

In some embodiments, Cas9, gRNA, and/or exogenous donor nucleic acids (e.g., ssODN) are delivered via AAV8. In some embodiments, Cas9, gRNA, and/or exogenous donor nucleic acids (e.g., ssODN) are delivered via AAV2/8. In some embodiments, Cas9, gRNA, and/or exogenous donor nucleic acids (e.g., ssODN) are delivered via an AAV strain comprising hybrid capsids with liver tropism.

In some embodiments, the modification of the endogenous rodent B4galt1 gene can be made specific to the liver through liver-specific expression of at least one component of the gene editing system. In some embodiments, the liver-specific expression is achieved by operably linking at least one component of the gene editing system components (e.g., in the case of a CRISPR/Cas system: gRNA, the Cas protein, the exogenous donor nucleic acid, etc.) to a liver-specific promoter. In a specific embodiment, the liver-specific promoter is an albumin promoter.

In some embodiments, specific liver targeting is facilitated by hydrodynamic tail vein injection of the components of the gene editing system. Methods for hydrodynamic tail vein injection are described in Kim, Mee J., and Nadav Ahituv. (Pharmacogenomics. Humana Press, Totowa, N.J., 2013. 279-289), herein incorporated by reference in its entirety.

In some embodiments, a combination of one or more of the embodiments described above is utilized to achieve liver-specific modification, i.e., a combination of (i) a liver tropic AAV system (to deliver one or more components of the CRISPR/Cas system or an exogenous donor nucleic acid, (ii) a liver-specific promoter to effect liver-specific expression of one or more components of the CRISPR/Cas system or an exogenous donor nucleic acid, and (iii) hydrodynamic tail vein injection of one or more components of the CRISPR/Cas system and an exogenous donor nucleic acid, or nucleic acid or viral vectors carrying the one or more components of the CRISPR/Cas system and an exogenous donor nucleic acid.

In some embodiments, a modification in a rodent B4galt1 gene is introduced into the genome (i.e., germline genome) of a rodent. This can be achieved by introducing a modification into a rodent B4galt1 gene in a rodent ES cell, then use a modified ES cell (i.e., a rodent ES cell having a modified rodent B4galt1 gene) as a donor cell to make a rodent having the modification in the germline genome.

In some embodiments, a modification is introduced into a rodent B4galt1 gene in a rodent ES cell by utilizing a gene editing system, as described above.

In some embodiments, a modification is introduced into an endogenous B4galt1 gene in a rodent ES cell through the use of a targeting vector which carries a rodent B4galt1 nucleic acid sequence containing the modification. The targeting vector can include, in addition to a modification-containing rodent B4galt1 nucleic acid sequence, flanking nucleic acid sequences that are of suitable lengths and homologous to rodent B4galt1 gene sequences at an endogenous rodent B4galt1 locus so as to be capable of mediating homologous recombination and integration of the mutation-containing rodent B4galt1 nucleic acid sequence into the endogenous rodent B4galt1 gene.

In some embodiments, a nucleic acid molecule (e.g., an insert nucleic acid) comprising a rodent B4galt1 gene modification is inserted into a vector, preferably a DNA vector. Depending on size, a modified rodent B4galt1 gene sequence can be cloned directly from cDNA sources or designed in silico based on published sequences available from GenBank. Alternatively, bacterial artificial chromosome (BAC) libraries can provide rodent B4galt1 gene sequences. Rodent B4galt1 gene sequences may also be isolated, cloned and/or transferred from yeast artificial chromosomes (YACs).

In some embodiments, the insert nucleic acid also contains a selectable marker gene (e.g., a self deleting cassette containing a selectable marker gene, as described in U.S. Pat. Nos. 8,697,851, 8,518,392 and 8,354,389, all of which are incorporated herein by reference), which can be flanked by or comprises site-specific recombination sites (e.g., loxP, Frt, etc.). The selectable marker gene can be placed on the vector adjacent to the mutation to permit easy selection of transfectants.

In some embodiments, a BAC vector carrying a modified rodent B4galt1 gene sequence can be introduced into rodent embryonic stem (ES) cells by, e.g., electroporation. Both mouse ES cells and rat ES cells have been described in the art. See, e.g., U.S. Pat. Nos. 7,576,259, 7,659,442, 7,294,754, and US 2008-0078000 A1 (all of which are incorporated herein by reference) describe mouse ES cells and the VELOCIMOUSE® method for making a genetically modified mouse; and US 2014/0235933 A1 and US 2014/0310828 A1 (all of which are incorporated herein by reference) describe rat ES cells and methods for making a genetically modified rat.

Homologous recombination in recipient cells can be facilitated by introducing a break in the chromosomal DNA at the integration site, which may be accomplished by targeting certain nucleases to the specific site of integration. DNA-binding proteins that recognize DNA sequences at the target locus are known in the art. In some embodiments, zinc finger nucleases (ZFNs), which recognize a particular 3-nucleotide sequence in a target sequence, are utilized. In some embodiments, Transcription activator-like (TAL) effector nucleases (TALENs) are employed for site-specific genome editing. In other embodiments, RNA-guided endonucleases (RGENs), which consist of components (Cas9 and tracrRNA) and a target-specific CRISPR RNA (crRNA), are utilized.

In some embodiments, a targeting vector carrying a nucleic acid of interest (e.g., a nucleic acid containing a modification to be introduced), flanked by 5 and 3′ homology arms, is introduced into a cell with one or more additional vectors or mRNA. In one embodiment, the one or more additional vectors or mRNA contain a nucleotide sequence encoding a site-specific nuclease, including but not limited to a zinc finger nuclease (ZFN), a ZFN dimer, a transcription activator-like effector nuclease (TALEN), a TAL effector domain fusion protein, and an RNA-guided DNA endonuclease.

ES cells having a modified gene sequence integrated in their genome, either through the use of a targeting vector or a gene editing system described, can be selected. After selection, positive ES clones can be modified, e.g., to remove a self-deleting cassette, if desired. ES cells having a modification integrated in the genome are then used as donor ES cells for injection into a pre-morula stage embryo (e.g., 8-cell stage embryo) by using the VELOCIMOUSE® method (see, e.g., U.S. Pat. Nos. 7,576,259, 7,659,442, 7,294,754, and US 2008/0078000 A1), or methods described in US 2014/0235933 A1 and US 2014/0310828 A1. The embryo comprising the donor ES cells is incubated until blastocyst stage and then implanted into a surrogate mother to produce an F0 rodent fully derived from the donor ES cells. Rodent pups bearing the mutant allele can be identified by genotyping of DNA isolated from tail snips using a modification of allele (MOA) assay (Valenzuela el al., supra) that detects the presence of the mutant sequence or a selectable marker gene.

In some embodiments, a modification is introduced into a rodent B4galt1 gene in a target tissue or organ of a rodent, instead of into the germline genome of a rodent.

In some embodiments, a modification is introduced into a rodent B4galt1 gene in the liver of a rodent, i.e., introduced specifically to the liver of the rodent. The term “liver-specific” means that the desired outcome (delivery, expression, and/or targeted modification) occurs significantly more (e.g., at least 25%, at least 50%, at least 75%, at least 100%, at least 150%, at least 200%, or greater), as compared to other tissues or organs. As described above, one or more of the following approaches can be utilized to achieve liver-specific modification: (i) a liver tropic AAV system (to deliver one or more components of the CRISPR/Cas system or an exogenous donor nucleic acid, (ii) a liver-specific promoter to effect liver-specific expression of one or more components of the CRISPR/Cas system or an exogenous donor nucleic acid, and (iii) hydrodynamic tail vein injection of one or more components of the CRISPR/Cas system and an exogenous donor nucleic acid, or nucleic acid or viral vectors carrying the one or more components of the CRISPR/Cas system and an exogenous donor nucleic acid.

### Methods of Breeding and Progenies Produced

In some embodiments, provided herein is a method that comprises breeding a first rodent whose genome comprises a modification in an endogenous rodent B4galt1 gene (i.e., a modified rodent B4galt1 gene) as disclosed herein, with a second rodent, resulting in a progeny rodent whose genome comprises the modification in the rodent B4galt1 gene. In some embodiments, the modification in an endogenous rodent B4galt1 gene results in a modified rodent B4galt1 gene which encodes a B4galt1 protein with reduced galactosyltransferase activity. In some embodiments, the modification in an endogenous rodent B4galt1 gene results in a modified rodent B4galt1 gene which encodes a B4galt1 protein comprising a substitution of Asn to Ser at an amino acid position corresponding to position 352 in a human B4GALT1 protein (i.e., “an N352S knock-in”); in such embodiments, the method comprises breeding a first rodent whose genome comprises an N352S knock-in with a second rodent, resulting in a progeny rodent whose genome comprises the N352S knock-in. Breeding (or “cross”, or “cross-breeding”) can be done following protocols readily available in the art; see, e.g., JoVE Science Education Database. Lab Animal Research, Fundamentals of Breeding and Weaning, JoVE, Cambridge, Mass., (2018) (video article); Breeding Strategies for Maintaining Colonies of Laboratory Mice. A Jackson Laboratory Resource Manual, ©2007 The Jackson Laboratory; all incorporated herein by reference.

In some embodiments, provided herein is a rodent progeny obtained from a breeding between a first rodent whose genome comprises a modification in an endogenous rodent B4galt1 gene as disclosed herein, with a second rodent. In some embodiments, the modification in an endogenous rodent B4galt1 gene results in a modified rodent B4galt1 gene which encodes a B4galt1 protein with reduced galactosyltransferase activity. In some embodiments, the modification in an endogenous rodent B4galt1 gene comprises an N352S knock-in; in such embodiment, a progeny is provided which comprises an N352S knock-in and is obtained from a breeding between a first rodent whose genome comprises an N352S knock-in with a second rodent. In some embodiments, the progeny rodent is heterozygous for the modification in the rodent B4galt1 gene. In some embodiments, the progeny rodent is homozygous for the modification in the rodent B4galt1 gene. The progeny may possess other desirable phenotypes or genetic modifications inherited from the second rodent used in the breeding.

### Rodent Model

In a further aspect, disclosed herein is use of a rodent which comprises a modification in an endogenous B4galt1 gene as an animal model, which permits elucidation of the function of B4galt1 in lipid metabolism and provides opportunities to test and develop therapeutics to target B4galt1 in the treatment of metabolic and cardiovascular disorders.

In some embodiments, a rodent which comprises a modification in an endogenous B4galt1 gene, as described herein, is used in a method of testing, screening, or identifying an agent that inhibits the activity of a B4galt1 protein. In some embodiments of the method, a rodent comprises a modification in an endogenous B4galt1 gene is used along with a wild type rodent without the modification, and a candidate agent is administered to the wild type rodent. Both the rodent with the modification and the wild type rodent are examined to measure their lipid profiles, for example, levels of HDL-C, LDL-C, and triglycerides. The measurements from the wild type rodent after the administration of the agent, from the wild type rodent before the administration (or from another wild type rodent not administered with the agent), and from the rodent with a modification in an endogenous B4galt1 gene, are compared with one another to determine whether the agent inhibits the activity of a B4galt1 protein. For instance, when the modification in an endogenous t4galt1 gene is a N352S knock-in or a loss of function mutation, an agent that results in a decreased level of LDL-C relative to the wild type rodent before the administration (or another wild type rodent not administered the agent (i.e., in the same direction as the rodent with the N352S knock-in or the loss of function mutation), is considered to inhibit the activity of a B4galt1 protein. In some embodiments, an agent results in a decrease in the serum LDL-C level in a wild type rodent administered with the agent by at least 10%, at least 15%, at least 20%, at least 25%, or more, relative to a wild type rodent not administered the agent.

In some embodiments, a rodent homozygous for a modification in an endogenous B4galt1 gene is used. In some embodiments, a rodent heterozygous for a modification on in an endogenous B4galt1 gene is used. In some embodiments, both a rodent homozygous for a modification in an endogenous B4galt1 gene, and a rodent heterozygous for a modification in an endogenous 4galt1 gene, are used in the examination.

In some embodiments, the rodent having a modification in an endogenous B4galt1gene rodent is a female. In some embodiments, the rodent having a modification in an endogenous B4galt1 gene is a male rodent.

A variety of candidate agents can be tested using the rodent and methods disclosed herein, including both small molecule compounds and large molecules (e.g., antibodies). In some embodiments, a candidate agent is an antibody specific for a B4galt1 protein (e.g., a human B4GALT1 protein). The present description is further illustrated by the following examples, which should not be construed as limiting in any way. The contents of all cited references (including literature references, issued patents, and published patent applications as cited throughout this application) are hereby expressly incorporated by reference.

The following representative embodiments are presented.

Embodiment 1. A rodent, comprising a modification in an endogenous rodent 04 galactotransferase 1 (B4galt1) gene at an endogenous rodent B4galt1 locus.
Embodiment 2. The rodent of embodiment 1, wherein the modification results in a modified rodent B4galt1 gene which encodes a B4galt1 protein comprising a substitution of Asn to Ser at an amino acid position corresponding to position 352 in a human B4GALT1 protein.
Embodiment 3. The rodent of embodiment 2, wherein the rodent is a mouse, and the substitution is at amino acid position 353 of a mouse B4galt1 protein.
Embodiment 4. The rodent of embodiment 2 or 3, wherein the rodent displays a decreased level of LDL-C, as compared to a wild type rodent without the modification.
Embodiment 5. The rodent of embodiment 1, wherein the modification is in the genome of the rodent.
Embodiment 6. The rodent according to any of embodiments 2-5, wherein the rodent is homozygous for the modification.
Embodiment 7. The rodent of embodiment 1, wherein the modification is a loss of function mutation.
Embodiment 8. The rodent of embodiment 7, wherein the loss of function mutation comprises an insertion, deletion or substitution of one or more nucleotides resulting in a deletion, in whole or in part, of the coding sequence of the endogenous rodent B4galt1 gene.
Embodiment 9. The rodent of embodiment 8, wherein the insertion, deletion or substitution of one or more nucleotides occurs in exon 2 of the endogenous rodent B4galt1 gene.
Embodiment 10. The rodent according to any one of embodiments 7-9, wherein the modification is in the genome of the rodent.
Embodiment 11. The rodent according to any one of embodiments 7-9, wherein the modification is introduced to the endogenous rodent B4galt1 gene in a target tissue or organ of the rodent.
Embodiment 12. The rodent of embodiment 11, wherein the modification is introduced to the endogenous rodent B4galt1 gene in the liver of the rodent.
Embodiment 13. The rodent according to any one of embodiments 1-2 or 4-12, wherein the rodent is a mouse or a rat.
Embodiment 14. An isolated rodent cell or tissue, comprising a modification in an endogenous rodent B4galt1 gene at an endogenous rodent B4galt1 locus.
Embodiment 15. The isolated rodent cell or tissue of embodiment 14, wherein the modification results in a modified rodent B4galt1 gene which encodes a B4galt1 protein comprising a substitution of Asn to Ser at an amino acid position corresponding to position 352 in a human B4GALT1 protein.
Embodiment 16. The isolated rodent cell or tissue of embodiment 15, wherein the rodent cell or tissue is a mouse cell or tissue, and the substitution is at amino acid position 353 of a mouse B4galt1 protein.
Embodiment 17. The isolated rodent cell or tissue of embodiment 14, wherein the modification is a loss of function mutation.
Embodiment 18. The isolated rodent cell or tissue of embodiment 17, wherein the loss of function mutation comprises an insertion, deletion or substitution of one or more nucleotides resulting in a deletion, in whole or in part, of the coding sequence of the endogenous rodent B4galt1 gene.
Embodiment 19. The isolated rodent cell or tissue of embodiment 18, wherein the insertion, deletion or substitution of one or more nucleotides occurs in exon 2 of the endogenous rodent B4galt1 gene.
Embodiment 20. The isolated rodent cell or tissue according to any one of embodiments 14-15 or 17-19, wherein the rodent cell or tissue is mouse cell or tissue.
Embodiment 21. The isolated rodent cell or tissue according to any one of embodiments 14-15 or 17-19, wherein the rodent cell or tissue is rat cell or tissue.
Embodiment 22. The isolated rodent cell or tissue according to any one of embodiments 14-21, wherein the rodent cell is a rodent embryonic stem (ES) cell.
Embodiment 23. A rodent embryo, comprising the isolated rodent cell of embodiment 22.
Embodiment 24. A method of making a genetically modified rodent, comprising

(i) introducing a modification into an endogenous rodent B4galt1 gene at an endogenous rodent B4galt1 locus of a rodent embryonic stem (ES) cell, thereby obtaining a modified rodent ES cell comprising a modified rodent B4galt1 gene; and

(ii) making the genetically modified rodent using the modified rodent ES cell.

Embodiment 25. The method of embodiment 24, wherein the modification results in a modified rodent B4galt1 gene which encodes a B4galt1 protein comprising a substitution of Asn to Ser at an amino acid position corresponding to position 352 in a human B4GALT1 protein.
Embodiment 26. The method of embodiment 25, wherein the rodent is a mouse, and the substitution is at amino acid position 353 of a mouse B4galt1 protein.
Embodiment 27. The method of embodiment 24, wherein the modification is a loss of function mutation.
Embodiment 28. The method of embodiment 27, wherein the loss of function mutation comprises an insertion, deletion or substitution of one or more nucleotides resulting in a deletion, in whole or in part, of the coding sequence of the endogenous rodent B4galt1 gene.
Embodiment 29. The method of embodiment 28, wherein the insertion, deletion or substitution of one or more nucleotides occurs in exon 2 of the endogenous rodent B4galt1 gene.
Embodiment 30. The method according to any one of embodiments 24-29, wherein the modification is introduced into the endogenous rodent B4galt1 gene through a gene editing system.
Embodiment 31. The method of embodiment 30, wherein the gene editing system is a CRISPR/Cas9 system.
Embodiment 32. The method of embodiment 31, wherein the gene editing system comprises a guide RNA, a Cas9 enzyme, and a single stranded oligodeoxynucleic acid molecule (ssODN).
Embodiment 33. The method of embodiment 32, wherein the guide RNA and ssODN are introduced into a rodent ES cell, wherein the rodent ES cell expresses the Cas9 enzyme.
Embodiment 34. A method of making a genetically modified rodent, comprising introducing a modification into an endogenous rodent B4galt1 gene at an endogenous rodent B4galt1 locus in a rodent tissue, thereby obtaining the genetically modified rodent.
Embodiment 35. The method of embodiment 34, wherein the modification results in a modified rodent B4galt1 gene which encodes a B4galt1 protein comprising a substitution of Asn to Ser at an amino acid position corresponding to position 352 in a human B4GALT1 protein.
Embodiment 36. The method of embodiment 35, wherein the rodent is a mouse, and the substitution is at amino acid position 353 of a mouse B4galt1 protein.
Embodiment 37. The method of embodiment 34, wherein the modification is a loss of function mutation.
Embodiment 38. The method of embodiment 37, wherein the loss of function mutation comprises an insertion, deletion or substitution of one or more nucleotides resulting in a deletion, in whole or in part, of the coding sequence of the endogenous rodent B4galt1 gene.
Embodiment 39. The method of embodiment 38, wherein the insertion, deletion or substitution of one or more nucleotides occurs in exon 2 of the endogenous rodent B4galt1 gene.
Embodiment 40. The method according to any one of embodiments 34-39, wherein the modification is introduced into the endogenous rodent B4galt1 gene through a gene editing system.
Embodiment 41. The method of embodiment 40, wherein the gene editing system is a CRISPR/Cas9 system.
Embodiment 42. The method of embodiment 41, wherein the CRISPR/Cas9 system comprises a guide RNA and a Cas9 enzyme, and wherein the guide RNA is delivered into the rodent by an AAV system.
Embodiment 43. The method of embodiment 42, wherein the AAV system targets delivery of the guide RNA into the liver of the rodent.
Embodiment 44. The method according to any one of embodiments 41-43, wherein the Cas9 enzyme is expressed in the rodent prior to introduction of the guide RNA into the rodent.
Embodiment 45. A rodent obtained by a method according to any one of embodiments 24-44.
Embodiment 46. A method of testing the effect of a compound on the activity of B4galt1, comprising

providing a rodent comprising a modification in an endogenous rodent B4galt1 gene according to any one of embodiments 1-13,

providing a wild type rodent without the modification,

administering a candidate B4galt1 inhibiting compound to the wild type rodent;

examining the rodent with the modification and the wild type rodent to measure serum LDL-C levels; and

comparing the measurements from the wild type rodent administered with the compound, from the wild type rodent before the administration of the compound, and from the rodent with the modification to determine whether the candidate compound inhibits the activity of B4galt1.

Embodiment 47. A method comprising breeding a first rodent whose genome comprises a modification in an endogenous rodent B4galt1 gene, with a second rodent.
Embodiment 48. The method of embodiment 47, wherein the modification results in a modified rodent B4galt1 gene which encodes a B4galt1 protein with reduced galactosyltransferase activity.
Embodiment 49. The method of embodiment 47, wherein the modification results in a modified rodent B4galt1 gene which encodes a B4galt1 protein comprising a substitution of Asn to Ser at an amino acid position corresponding to position 352 in a human B4GALT1 protein.
Embodiment 50. A progeny obtained from a method according to any one of embodiments 47-49, wherein the progeny comprises the modification in its genome.

### EXAMPLES

The following examples are put forth so as to provide those of ordinary skill in the art with a complete disclosure and description of how the compositions and/or methods claimed herein are made and evaluated, and are intended to be purely exemplary and are not intended to limit the disclosure.

### Example 1. Generation and Characterization of N352S K/I Mice

### Design and Generation of N352S K/I Mice

To make B4galt1 p.N353S mutant mice, CRISPR Cas9 gene editing technology was used. Briefly, 35 ug synthesized ssODN, 2.5 ug synthesized guide RNA (gRNA) and 5 ug Cas9 protein were electroporated into 100% C57BL/6NTac (VGB6) mouse embryonic stem cells (ESCs). The sequence of ssODN is ATGCTGTAGTAGGGAGGTGTCGAATGATCCGGCATTCAAGAGACAAGAAAAATG AGCCCAgTCCcCAGAGGTACGTCCTCTCTGTGCCTTCCCTTTATTTATTTATATGTT AGATTTATTT (SEQ ID: 13, the nucleotides in lower cases are the point mutations causing p.N353S and p.P354P nonsynonymous changes in the targeted ES clones). The sequence of gRNA is GAGGACGTACCTCTGAGGATtgg (SEQ ID NO: 11, the nucleotides in lower cases are PAM sequence). The targeted cells were screened by TaqMan qPCR assays and then microinjected into 8-cell embryos from Charles River Laboratories Swiss Webster albino mice, yielding F0 VelociMice® that were 100% derived from the targeted cells (Poueymirou et al., 2007, Nature Biotech. 25(1).91-99). F0 mice were bred once to C57BL/6NTac to generate targeted mice in a genetic background that was 100% C57BL/6NTac, which were subsequently bred to homozygosity and maintained in the Regeneron animal facility during the whole period. Wild-type mice used as controls in all experiments were also 100% C57BL/6NTac.

### Plasma Collection

Mice were kept on regular chow diet and they were bled at 13 weeks of age (Female WT=14, Het=16, HO=16; Male WT=15, Het=18, HO=14). Mice were fasted overnight and then anesthetized with 4.5% Isoflurane. After checking for a lack of pedal reflex, 150-200 ul of whole blood was obtained via retro-orbital sinus tap and immediately transferred into plasma collection tubes coated with K₃ EDTA (Starstedt) and containing protease (Roche cOmplete™ Mini EDTA Free) and DPP-4 inhibitors. Plasma was collected into an eppendorf tube and stored at −80° C.

Samples were assayed using the ADVIA Chemistry XPT (Siemens). The Liver Lipid Profile contains the following reagents: Alanine Aminotransferase (ALT)-(Siemens REF 03036926); Aspartate Aminotransferase (AST)-(Siemens REF 07499718); Cholesterol_2 (CHOL_2)-(Siemens REF 10376501); Direct HDL Cholesterol (D-HDL)-(Siemens REF 07511947); LDL Cholesterol Direct (DLDL)-(Siemens REF 09793248); Non-Esterified Fatty Acids (NEFA)-(Wako 999-34691, 995-34791, 991-34891, 993-35191); Triglycerides_2 (TRIG_2)-(Siemens REF 10335892). Samples were loaded into the analyzer and reagent mixing, assay timing, absorbance and concentration calculation was performed by the analyzer. Statistics were calculated using Two Way ANOVA with Sidak's Multiple Comparison test (Prism).

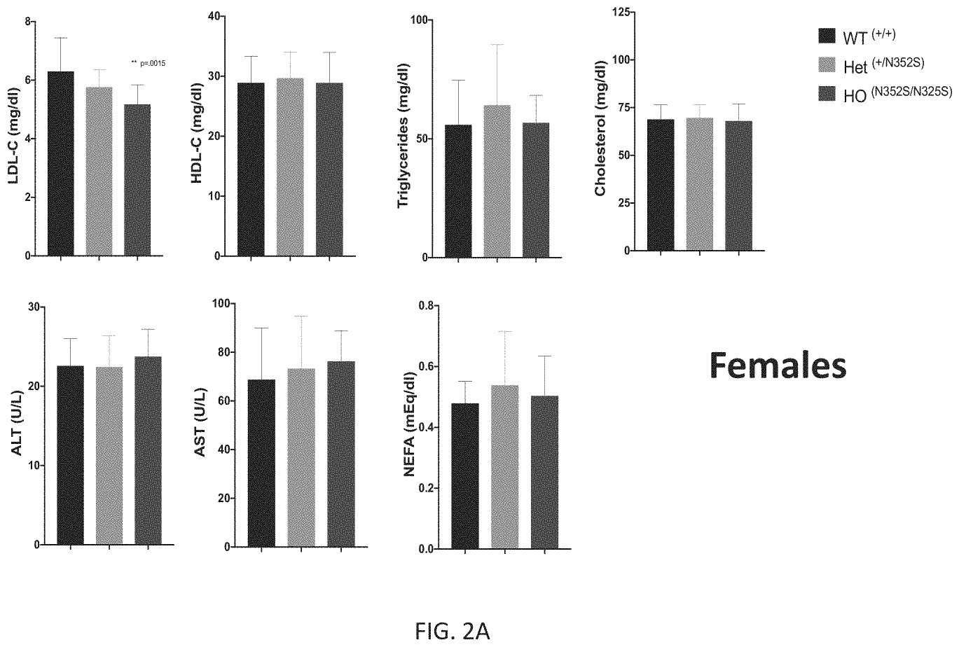

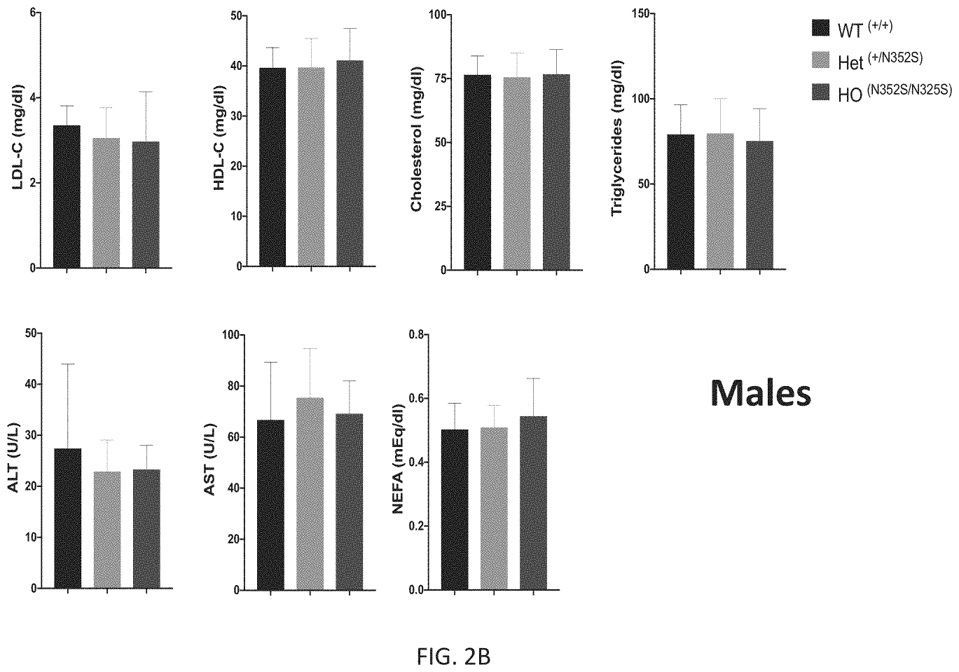

As shown in FIGS. 2A-2B, decreased levels of LDL-C were observed in heterozygous and homozygous N352S knock-in in both male and female mice, as compared to wild type mice, while no overt differences were observed for levels of HDL-C, triglycerides, cholesterol or Non-Esterified Fatty Acids (NEFA) between heterozygous or homozygous N352S knock-in mice and wild type mice. Further, no significant differences were observed for levels of ALT or AST between heterozygous or homozygous N352S knock-in mice and wild type mice.

### Example 2. Generation and Characterization of B4galt1 Liver Knock-Out Mice

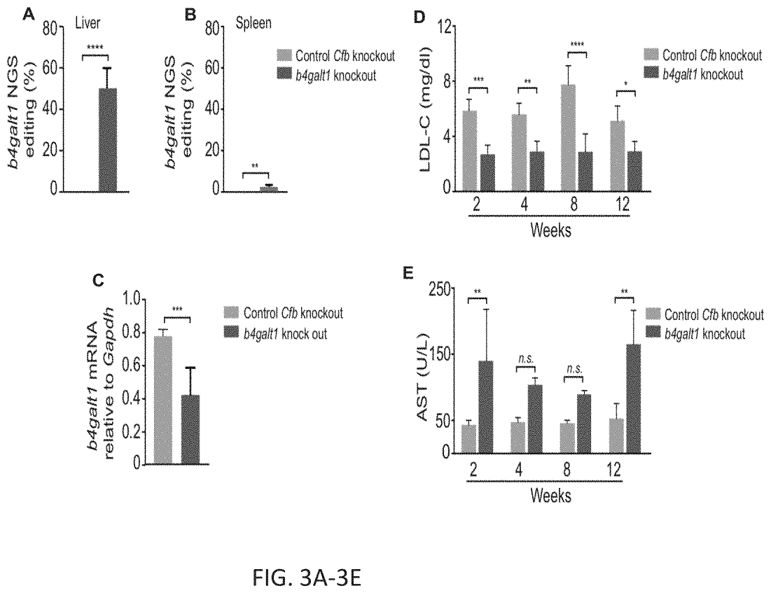

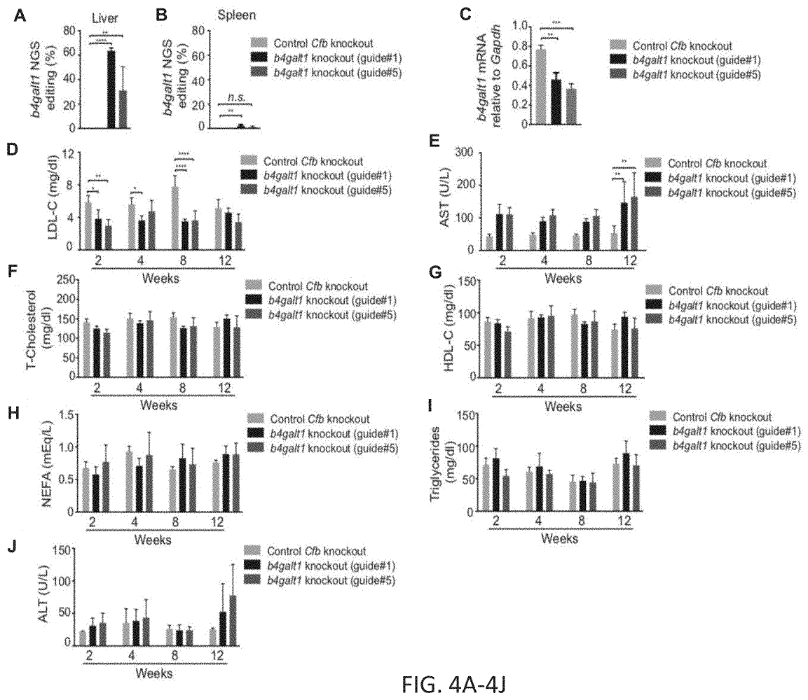

To further understand the role of B4GALT1 in lipid metabolism, the CRISPR/Cas9 in vivo toolbox was utilized to knock-out the mouse ortholog (B4galt1) in liver. Briefly, adult mice constitutively expressing Cas9 enzyme were transduced with an AAV8 to achieve a liver specific delivery of gRNAs targeting the exon 2 of B4galt1 (see Methods below). This approach resulted in a 50% gene editing of B4galt1 in the liver, versus an approximately 2% gene editing of B4galt1 in the spleen (FIGS. 3A, 3B). As a result, a 50% decrease of B4galt1 mRNA levels in liver were observed (FIG. 3C), with no changes of mRNA in the spleen (data not shown). Circulating LDL-C levels were then measured starting at 2 weeks from the viral transduction and throughout a 12-weeks study period. An overall 50% decrease of LDL-C was detected during the entire duration of the study (FIG. 3D; 2 weeks p<0.0001; 4 weeks p <0.01; 8 weeks p<0.00001; 12 weeks p<0.01). At the same time, a trend toward increased circulating AST enzyme was also observed over time, while no significant changes in HDL-C or total cholesterol were observed (FIG. 3E). The decrease in LDL-C levels was confirmed by using two other independent gRNAs always designed against exon2 of B4galt1 (FIGS. 4A-4J).

Finally, to ascertain that the decrease seen in the LDL-C was determined by B4galt1 specific ablation, a liver-specific knockout of a second independent gene, Cfb, (encoding complement factor B) was concomitantly generated. Cfb was chosen as control because of its high expression in liver and yet no function in LDL-C and cholesterol metabolism. This resulted in a roughly 50% editing rate of Cfb in the liver, versus an <1% editing in the spleen and a 50% decrease of Cfb hepatic expression. As expected, LDL-C levels were not affected by the Cfb liver knockout. Moreover, this approach enabled us to rule out any possible secondary effect from Cas9 constitutive expression and viral infection. Any variation of LDL-C levels in the Cfb control liver knockout over time was minimal and mainly due to the intrinsic variability of in vivo manipulation (FIG. 3D and FIGS. 4A-4J).

These results support a functional link between b4galt1 and LDL-C metabolism in a mammalian system.

### Methods

Generation of Cas9 mESC

Targeting of mESC (50% C57BL/6NTac and 50% 129S6/SvEvTac) was performed using previously described methods (Valenzuela et al., Nat Biotechnol, 2003. 21(6): p. 652-9). Briefly, a targeting vector was built by modifying the R26 BAC (BAC_ESr2-445b1_sfi_1) to replace part of R26 intron one with a cassette containing neomycin selection (amino 3′-glycosyl phosphotransferase) with tandem polyadenylation signals flanked by LoxP sites followed by Cas9 with a P2A GFP such that the transcript can be driven by the R26 promoter in mESCs. The linearized modified BAC was then electroporated into mESCs to drive homologous recombination at the R26 locus utilizing the targeting arms from the modified BAC. Positive transformants were selected for neomycin resistance. Transgenic insertions were distinguished from targeted recombination based on quantitative polymerase chain reaction (qPCR). Once targeting was confirmed, the clones were electroporated with Cre recombinase to excise the blocking cassette and generate the active alleles.

### Mouse Production

Cas9 mESCs were injected into eight-cell embryos to generate 100% ES derived F0 mice (Valenzuela et al., Nat Biotechnol, 2003. 21(6): p. 652-9; Poueymirou et al., Nat Biotechnol, 2007. 25(1): p. 91-9). Injected eight-cell embryos were transferred to surrogate mothers to produce live pups carrying the desired insertion. Upon gestation in a surrogate mother, the injected embryos produce F0 mice that carry no detectable host embryo contribution. The fully mESC-derived mice were generally normal, healthy, and fertile. All animal experiments were performed in accordance with the guidelines for the Institutional Animal Care and Use Committee (IACUC) at Regeneron.

### Design of Guide RNA

Guide RNAs were designed using UCSC (NCBI37/mm9) with reference to CRISPOR and BLAT (Kent et al., Genome Res, 2002. 12(6): p. 996-1006; Kent, Genome Res, 2002. 12(4): p. 656-64; Haeussler et al., Genome Biol, 2016. 17(1): p. 148). The Cas9 KO guides were designed to target B4galt1 exon 2: B4galt1_mGU1; TATTAAAGTCAATCAGCATG (SEQ ID NO: 7) at chr4:40770681-40770700, B4galt1_mGU3; GGGCGGCCGTTACTCCCCCA (SEQ ID NO: 8) at msChr4:40770612-40770631, and B4galt1_mGU5; ATGATGATGGCCACCTTGTG (SEQ ID NO: 9) at msChr4:40770575-40770594. Additionally, (fb was chosen as control, the Cas9 KO guides were designed to target GAGCGCAACTCCAGTGCTTG (SEQ ID NO: 10) at msChr17:34998886-34998905.

### Generation of Viral Particles

Guide RNA sequences were cloned into the appropriate AAV backbones by standard ligation. AAV8 vectors were produced by transient transfection of HEK 293T cells. Transfections were performed using Polyethylenimine (PEI) MAX (Polysciences). Cells were transfected with three plasmids encoding adenovirus helper genes, AAV2 rep and AAV8 cap genes, and recombinant AAV genomes containing transgenes flanked by AAV2 inverted terminal repeats (ITRs). Virus containing medium was collected and filtered through a 0.2 μm PES membrane (Nalgene). Virus was either purified by a series of centrifugation steps or density gradient ultracentrifugation.

For purification by centrifugation, virus containing medium was concentrated by PEG precipitation as previously described (Arden et al., J Biol Methods, 2016. 3(2)). The pellet was resuspended in PBS (Life Technologies) and further clarified by centrifugation at 10,000 RCF. The supernatant was transferred and the AAV was further pelleted in an ultracentrifuge at 149,600 RCF for 3 hours at 10° C. The AAV containing pellet was resuspended in PBS, clarified by centrifugation, and filtered through a 0.22 μm cellulose acetate membrane (Corning).

For purification by iodixanol gradient separation, medium was concentrated by tangential flow filtration and loaded onto an iodixanol gradient. Iodixanol solutions and gradients were prepared with slight modifications as previously described (Zolotukhin et al., Gene Ther, 1999. 6(6): p. 973-85). Gradients were spun at 149,600 RCF for 14 hours in an ultracentrifuge. The AAV containing fraction was extracted and buffer was exchanged into PBS with 0.001% Pluronic (ThermoFisher Scientific) using Zeba Spin Desalting columns (ThermoFisher Scientific).

### Tail Vein Injection

The lateral tail vein was injected by inserting a 27-gauge needle into the vein at the base of the tail and injecting approximately 2×10¹¹ viral genomes in 100 μL.

### Amplicon Library Prep

Liver and spleen biopsies were harvested from 3-5 study animals per treatment (AAV B4galt1 CR1-5; unrelated control) and genomic DNA (gDNA) was extracted using a proteinase K-based lysis buffer. Target specific oligos were designed (21-27 base pairs, bp) to generate a maximum amplicon size of 350 bp with primer melting temperature (Tm) of 60-65° C. degrees. Illumina adapters were added to the target specific oligo and the full sequence was ordered from Integrated DNA Technologies (IDT). Polymerase Chain Reaction (PCR) was completed on each gDNA sample. Briefly, in each reaction, 4 nanograms (ng) of gDNA was combined with IDT oligos, Q5 polymerase (#M0491, New England Biolabs), 10 uM dNTPs, buffer, and water per manufacturer's specifications. Next, the amplification products were diluted 1:100 and used for the PCR barcoding reaction to create the final sequencing library. Each barcoding reaction contained a single amplified target and forward and reverse primers with a unique, Illumina specific, barcode and index. Each plate of PCRs was pooled in equal volumes and then purified in a single tube using AMPure XP reagent (#A63881, Beckmann-Coulter), as per the manufacturer's instructions. Final library concentration was measured using the Qubit fluorometer (#Q32866, Invitrogen). Four nanomoles of the prepared library was loaded onto the Illumina MiSeq according to the manufacturer's instruction utilizing the 2×300 read kit (#MS-102-3003, Illumina).

### Sequence Mapping and Characterization

Barcoded samples were de-multiplexed to individual reads (FASTQ format). Forward and reverse reads of each FASTQ file were then merged using the PEAR program (described in Zhang et al., Bioinformatics. 2014 Mar. 1; 30(5): 614-620). Merged reads were mapped to the Mus musculus genome version 9 (mm9) using the Bowtie2 program (described in Langmead et al., Nat Methods. 2012 Mar. 4; 9(4): 357-359). Each sample was sequenced with a minimum of 20,000 merged reads across the expected guide cleavage location. Finally, characterization of barcoded samples was performed using a custom perl script. Briefly, all insertions, deletions, or base changes (INDEL) within a window of 20 bases upstream and downstream of the expected cut site were considered to be CRISPR induced modifications. The number of reads containing INDELs was compared to the number of reads with wild type sequence to determine the B4galt1 percent editing per animal and tissue.

### Taqman Expression Analysis

Liver was dissected fresh into RNALater stabilization reagent (Qiagen) and stored at −20° C. Tissues were homogenized in TRIzol and chloroform was used for phase separation. The aqueous phase, containing total RNA, was purified using miRNeasy Mini Kit (Qiagen, Cat #217004) according to manufacturer's specifications. Genomic DNA was removed using MagMAX™Turbo™DNase Buffer and TURBO DNase (Ambion by Life Technologies). mRNA (Up to 2.5 ug) was reverse-transcribed into cDNA using SuperScript® VILO™ Master Mix (Thermofisher). cDNA was amplified with the SensiFASY Probe Hi-ROX (Meridian) using the ABI 7900HT Sequence Detection System (Applied Biosystem). Gapdh was used as the internal control gene to normalize cDNA input differences.

### Plasma Collection

Mice were fasted overnight and then anesthetized with 4.5% Isoflurane. After checking for a lack of pedal reflex, 150-200 ul of whole blood was obtained via retro-orbital sinus tap and immediately transferred into plasma collection tubes coated with K₃ EDTA (Starstedt) and containing protease (Roche cOmplete™ Mini EDTA Free) and DPP-4 inhibitors. Plasma was collected into an eppendorf tube and stored at −80 C.

Samples were assayed using the ADVIA Chemistry XPT (Siemens). The Liver Lipid Profile contains the following reagents: Alanine Aminotransferase (ALT)-(Siemens REF 03036926); Aspartate Aminotransferase (AST)-(Siemens REF 07499718); Cholesterol_2 (CHOL_2)-(Siemens REF 10376501); Direct HDL Cholesterol (D-HDL)-(Siemens REF 07511947); LDL Cholesterol Direct (DLDL)-(Siemens REF 09793248); Non-Esterified Fatty Acids (NEFA)-(Wako 999-34691, 995-34791, 991-34891, 993-35191); Triglycerides_2 (TRIG_2)-(Siemens REF 10335892). Samples were loaded into the analyzer and reagent mixing, assay timing, absorbance and concentration calculation was performed by the analyzer. Statistics were calculated using Two Way ANOVA with Sidak's Multiple Comparison test (Prism).

1. A rodent, comprising a modification in an endogenous rodent p4 galactotransferase 1 (B4galt1) gene at an endogenous rodent B4galt1 locus.

2. The rodent of claim 1, wherein the modification results in a modified rodent B4galt1 gene which encodes a B4galt1 protein comprising a substitution of Asn to Ser at an amino acid position corresponding to position 352 in a human B4GALT1 protein.

3. The rodent of claim 2, wherein the rodent is a mouse, and the substitution is at amino acid position 353 of a mouse B4galt1 protein.

4. The rodent of claim 2, wherein the rodent displays a decreased level of LDL-C, as compared to a wild type rodent without the modification.

5. The rodent of claim 1, wherein the modification is in the genome of the rodent.

6. The rodent of claim 2, wherein the rodent is homozygous for the modification.

7. The rodent of claim 1, wherein the modification is a loss of function mutation.

8. The rodent of claim 7, wherein the loss of function mutation comprises an insertion, deletion or substitution of one or more nucleotides resulting in a deletion, in whole or in part, of the coding sequence of the endogenous rodent B4galt1 gene.

9. The rodent of claim 8, wherein the insertion, deletion or substitution of one or more nucleotides occurs in exon 2 of the endogenous rodent B4galt1 gene.

10. The rodent of claim 7, wherein the modification is in the genome of the rodent.

11. The rodent of claim 7, wherein the modification is introduced to the endogenous rodent B4galt1 gene in a target tissue or organ of the rodent.

12. The rodent of claim 11, wherein the modification is introduced to the endogenous rodent B4galt1 gene in the liver of the rodent.

13. The rodent of claim 1, wherein the rodent is a mouse or a rat.

14. An isolated rodent cell or tissue, comprising a modification in an endogenous rodent B4galt1 gene at an endogenous rodent B4galt1 locus.

15.-21. (canceled)

22. The isolated rodent cell or tissue of claim 14, wherein the rodent cell is a rodent embryonic stem (ES) cell.

23. A rodent embryo, comprising the isolated rodent cell of claim 22.

24. A method of making a genetically modified rodent, comprising
(i) introducing a modification into an endogenous rodent B4galt1 gene at an endogenous rodent B4galt1 locus of a rodent embryonic stem (ES) cell, thereby obtaining a modified rodent ES cell comprising a modified rodent B4galt1 gene; and
(ii) making the genetically modified rodent using the modified rodent ES cell.

25.-33. (canceled)

34. A method of making a genetically modified rodent, comprising introducing a modification into an endogenous rodent B4galt1 gene at an endogenous rodent B4galt1 locus in a rodent tissue, thereby obtaining the genetically modified rodent.

35.-41. (canceled)

42. The method of claim 34, wherein the modification is introduced into the endogenous rodent B4galt1 gene through a CRISPR/Cas9 system comprising a guide RNA and a Cas9 enzyme, and wherein the guide RNA is delivered into the rodent by an AAV system.

43. The method of claim 42, wherein the AAV system targets delivery of the guide RNA into the liver of the rodent.

44.-46. (canceled)
